(19) United States
(12) Patent Application Publication
(10) Pub. No.: US 2003/0124146 A1
(43) Pub. Date: Jul. 3, 2003

(54) Recombinant Rhabdoviruses as live-viral vaccines

(76) Inventors: Matthias J. Schnell, Harleysville, PA (US); Roger J. Pomerantz, Chalfont, PA (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/150,165

(22) Filed: May 16, 2002

Related U.S. Application Data
(63) Continuation-in-part of application No. 09/494,262, filed on Jan. 28, 2000, now abandoned.
(63) Continuation-in-part of application No. 09/761,312, filed on Jan. 17, 2001.
(60) Provisional application No. 60/291,349, filed on May 16, 2001.
(60) Provisional application No. 60/285,552, filed on Apr. 20, 2001.

(51) Int. Cl. A61K 39/205; C07H 21/04; C12N 7/00; C12N 15/86

(52) U.S. Cl. 424/224.1; 435/235.1; 536/23.72; 435/456; 435/320.1

(57) ABSTRACT

This invention provides recombinant, replication-competent Rhabdovirus vaccine strain-based expression vectors for expressing heterologous viral antigenic polypeptides such as immunodeficiency virus envelope proteins or subparts thereof. An additional transcription stop/start unit within the Rhabdovirus genome is inserted to express the heterologous antigenic polypeptides. The HIV-1 gp160 protein is stably and functionally expressed, as indicated by fusion of human T cell-lines after infection with the recombinant RVs. Inoculation of mice with the recombinant Rabies viruses expressing HIV-1 gp160 induces a strong humoral response directed against the HIV-1 envelope protein after a single boost with an isolated recombinant HIV-1 gp120 protein. Moreover, high neutralization titers, up to 1:800, against HIV-1 are detected in the mouse sera. These recombinant viral vectors expressing viral antigenic polypeptides provide useful and effective pharmaceutical compositions for the generation of viral-specific immune responses.

### CROSS REFERENCE TO RELATED APPLICATIONS

This application claims priority under 35 U.S.C. §120 based upon U.S. Provisional Application No. 60/291,349 filed May 16, 2001. This application claims priority, in part, under 35 U.S.C. §120 based upon U.S. Non-Provisional Application Reference Number SCH02-CP201 filed Apr. 19, 2002 which is a continuation-in-part of 09/494,262 filed Jan. 28, 2000. This application is a continuation-in-part of U.S. Non-Provisional application Ser. No. 09/761,312, filed Jan. 17, 2001. This application claims priority, in part, under 35 U.S.C. §119 based upon U.S. Provisional Application No. 60/285,552, filed Apr. 20, 2001.

### GOVERNMENT RIGHTS TO THE INVENTION

This invention was made in part with government support under grant AI44340 awarded by the National Institute of Health. The government has certain rights to the invention.

### FIELD OF THE INVENTION

The present invention relates to the fields of molecular biology and virology, and to a method of treating an HIV-1 infection and to a method of treating an HCV infection, more particularly, to the induction of both humoral and cellular immunity against HIV-1 and against HCV.

### BACKGROUND OF THE INVENTION

Great success has been made in the therapy of HIV-1 infection during the last several years. (Holtzer, et al., Annals of Pharmacotherapy 33:198-209, 1999; Bonfanti, et al., Biomedicine & Pharmacotherapy, 53:93-105, 1999). However, the development of a protective immunodeficiency virus vaccine (e.g., HIV-1 vaccine) still remains a major goal in halting immunodeficiency virus pandemics. Most successful vaccines against viral diseases have been composed of killed or attenuated viruses. (Hilleman, M. R., Nature Medicine, 4:507-14, 1998). This approach does not seem to be suitable for immunodeficiency viruses, particularly HIV-1 because killed HIV-1 virus induces only a poor neutralizing antibody response and no cytotoxic T lymphocyte (CTL) response.

New anti-retroviral strategies against human HIV-1 result in a dramatic decrease in mortality among infected humans in developed countries, but the development of a successful vaccine to prevent infection is still the major goal to halt the HIV-1 pandemic. A human being is infected with HIV-1 every 10 seconds on average, and in the heavily affected countries in Africa, such as Zambia and Uganda, nearly 40% of young adults are HIV-1-seropositive.

Currently, a variety of HIV vaccine strategies are being investigated, including recombinant proteins (Goebel, F. D., et al., European Multinational IMMUNO AIDS Vaccine Study Group Aids, 5:643-50, 1999; Quinnan, G. V., Jr., et al., AIDS Research & Human Retroviruses, 15:561-70, 1999; VanCott, T. C., et al., J. Virol., 73:4640-50, 1999), peptides (Bekyakov, I. M., et al., Journal of Clinical Investigation, 102:2072-81, 1998; Berzofsky, J. A., et al., Immunological Reviews, 170:151-72. 1999; Pinto, L. A., et al., AIDS, 13:2003-12, 1999), naked DNA (Bagarazzi, M. L., et al., 1999, Journal of Infectious Diseases, 180:1351-5, 1999; Barouch, D. H., et al., Science, 290:486-492, 2000; Cafaro, A., et al., Nature Medicine, 5:643-50, 1999; Lu, S., et al., AIDS Research & Human Retroviruses, 14:151-5, 1998; Putkonen, P., et al., Virology, 250:293-301, 1998; Robinson, H. L., Aids, 11:S 109-19, 1997; Weiner, D. B., and R. C. Kennedy, Scientific American, 281:50-7, 1999.), replication-competent and incompetent (replicon) live viral vectors (Berglund, P., et al., AIDS Research & Human Retroviruses, 13:1487-95, 1997; Mossman, S. P., et al., J. Virol., 70:1953-60, 1996; Natuk, R. J., et al., Proc. Natl. Acad. Sci. USA, 89:7777-81, 1992; Ourmanov, I., et al., J. Virol., 74:2740-2751, 2000; Schnell, M. J., et al., Proc. Natl. Acad. Sci. USA, 97:3544-3549, 2000.), and prime-boost combinations. [for review see (5)]. A large number of these vaccine strategies have been tested in the simian immunodeficiency virus (SIV) macaque model system, but to date no potent protective immunity has been obtained, although some amelioration of disease course has been seen. (Barouch, D. H., et al., Science, 290:486-492, 2000; Davis, N. L., et al., J. Virol, 74:371-8, 2000; Ourmanov, L, et al., J. Virol., 74:2740-2751, 2000.). So far, the only effective method to protect macaques from SIV infection is the use of live, attenuated SIV. Desrosiers and colleagues showed that a genetically modified, nef-deleted SIV strain that does not cause disease in rhesus monkeys induced high anti-SIV titers of antibodies and cytotoxic T lymphocyte (CTL) activity. (Daniel, M. D., et al., Science, 258, 1938-1941, 1992; Kestler, H. W., et al., Cell, 65:651-662, 1991.). Subsequent challenge of the immunized animals with infectious doses of a pathogenic SIV strain yielded protection from infection. (Daniel, M. D., et al., Science, 258:1938-1941, 1992). A major drawback in the use of attenuated lentiviral vaccine approaches is the finding that even nef-deleted SIV can give rise to an AIDS-like disease in both neonatal and adult macaques. (Baba, T. W., et al., Science, 267:1820-5, 1995; Baba, T. W., et al., Nature Medicine, 5:194-203, 1999; Desrosiers, R. C., AIDS Research & Human Retroviruses, 10:331-2, 1994.). Additional concerns regarding the use of attenuated lentiviruses arise from the recent finding that recombination of live, attenuated SIV with challenge virus in some cases results in an even more virulent strain. (Gundlach, B. R., et al., J. Virol., 74:3537-3542, 2000.). However, the results indicated that live-viral vectors may be excellent vaccine candidates for an HIV-1 20 vaccine.

For the foregoing reasons, there is a great need for the development of a protective immunodeficiency virus vaccine that is non-pathogenic for a wide range of animal species when administered orally or intramuscularly, as well as being able to induce the required neutralizing antibody and CTL responses.

The immune response(s) required to protect against HIV-1 infection is currently unknown, but a protective immune response against HIV-1 might require both major arms of the immune systems. Recent reports on vaccine approaches using recombinant HIV-1 envelope protein suggests that an exclusively humoral response is not sufficient to protect against an HIV-1 infection, but the passive transfer of three monoclonal antibodies directed against HIV-1 envelope protein resulted in protection of macaques against subsequent challenge with pathogenic HIV-1/SIV chimeric virus. (Mascola, J. R., et al., Nature Medicine, 6:207-10, 2000.). Other studies indicate that a cell-mediated response plays an important role in controlling an HIV-1 infection. (Brander, C. and B. D. Walker, Current Opinion in Immunology, 11:451-9 1999; Goulder, P. J., et al., Anti-HIV cellular immunity: recent advances towards vaccine design Aids, 13:S121-36, 1999.). Exposed but uninfected individuals often have HIV-1-specific CTLs but no detectable antibodies against HIV-1 (Pinto, L. A., et al., Journal of Clinical Investigation, 96:867-76, 1995; Rowland-Jones, S. L., et al., Journal of Clinical Investigation, 102:1758-65, 1998.).

In the present invention, the ability of recombinant non-segmented negative-stranded RNA viruses expressing an immunodeficiency virus gene(s) as an immunodeficiency virus vaccine (e.g., HIV-1 vaccine) is disclosed. Specifically, the ability of a Rhabdovirus-based recombinant viruses to induce an immune response against HIV is demonstrated. The HIV-1 envelope protein is stably and functionally expressed and induces a strong humoral response directed against the HIV-1 envelope protein after a single boost with recombinant HIV-1 protein boost (gp120) in mice. Moreover, high neutralization titers against HIV-1 are detected in the mouse sera. (Schnell, M. J., et al., Proc. Natl. Acad. Sci. USA, 97:3544-3549, 2000.).

Little information is available regarding the induction of CTL responses against foreign proteins expressed by rhabdovirus-based vectors. Growing evidence suggests that a CD8+ cytotoxic T-cell (CTL)-mediated immune response is critical in controlling HIV-1 infection (21, 37). This finding is based on several studies showing that exposed but uninfected individuals have HIV-1-specific CTLs without detectable antibodies (44, 45). In addition, data show a strong correlation between a high frequency of HIV-1 specific CTLs with a low HIV-1 viral titer and a slow disease progression in chronically HIV-1-infected individuals. Furthermore, the elimination of CD8+ lymphocytes from monkeys during chronic simian immunodeficiency virus (SIV) infection resulted in a rapid and marked increase in viremia that was suppressed coincident with the reappearance of SIV-specific CD8+ T cells (46).

The present invention fulfills this long sought need for an efficacious HIV-1 vaccine. The present invention relates to recombinant RV vaccines expressing HIV-1 envelope proteins to induce HIV-1-specific CTLs. Specifically, a single inoculation of the HIV-1 virus vaccines of the present invention induce a solid and long-lasting memory CTL response specific for HIV-1 proteins. These recombinant viruses are non-pathogenic for a wide range of animal species when administered orally or intramuscularly. In a specific embodiment when the coding region of the HIV-1 gp160 (strains NL4-3 and 89.6) is cloned between the RV glycoprotein (G) and polymerase (L) proteins under the control of a RV transcription Stop/Start signal, the resulting recombinant RVs expressed HIV-1 gp160 along with the other RV proteins. In another embodiment, the present invention provides a replication-competent RV vaccine strain-based vector expressing HIV-1 Gag to elicit an HIV-1 Gag-specific CTL response in mice. A single inoculation of either vector results in a vigorous CTL response that is specific for HIV-1, thereby providing an efficacious HIV-1 vaccine.

In addition to HIV, treatment regimens targeted against Hepatitis C virus (HCV), the primary etiological agent of non-A, non-B hepatitis, are expensive, show a relatively low rate of response, and carry the potential for significant side effects (Fried and Hoofnagle, 1995). The majority of patients (70%) with HCV develop chronic hepatitis and a third of these cases progress to liver cirhossis. All infected individuals have an increased risk of hepatocellular carcinoma (Aihara and Miyazaki, 1998). Therefore, there is a long sought, yet unfulfilled need for the development of a protective vaccine against HCV. The present invention fulfills this need by providing a Rhabdovirus-based recombinant virus vaccine expressing HCV glycoprotein(s) to induce an immune response against HCV.

HCV is a small, enveloped positive strand RNA virus of the Flaviviridae family (Clarke, 1997). The 9.6 kilobase genome consists of a 5′ nontranslated region (NTR) which contains an internal ribosome entry site (IRES) to begin translation of the viral polyprotein (Le, Siddiqui, and Maizel, 1996), which is cleaved by both host and viral proteases to yield four structural and six non-structural (NS) proteins (Reed and Rice, 1998). The genome encodes two envelope glycoproteins, E1 and E2, which are released from the polyprotein via signal peptidase cleavages (Grakoui et al., 1993). Both proteins are largely modified by N-linked glycosylation and are thought to be type I integral transmembrane proteins with C-terminal hydrophobic anchor domains. Expression of both the glycoproteins in mammalian cell-lines illustrates their retention in the endoplasmic reticulum (ER), with no surface expression detectable (Duvet et al., 1998).

The E2 glycoprotein contains two hypervariable regions (HVR), with HVR1 located at amino acid positions 390-410, and HVR2 located at positions 474-480 (Weiner et al., 1991). Antibodies directed against the HVR1 of E2 have been implicated in controlling HCV infection (Kato et al., 1993). In addition, the HVR1 of E2 contains both B-cell and cytotoxic T-lymphocyte (CTL) epitopes. Furthermore, antibodies specifically directed at this region reportedly blocked viral attachment in susceptible cells, further implicating E2 as responsible for viral attachment to the host cell (Kojima et al., 1994; Leroux-Roels et al., 1996; Lesniewski et al., 1995).

To date, HCV vaccine studies involving E2 have utilized several strategies in a murine model including purified recombinant antigens, DNA immunization (Gordon et al., 2000), DNA priming in conjunction with recombinant viruses such as Semliki Forest Virus and canarypox (Pancholi et al., 2000; Vidalin et al., 2000), DNA priming with recombinant protein boosting (Song et al., 2000), replication-deficient recombinant adenovirus (Makimura et al., 1996), and plasmid DNA immunization (Inchauspe, 1999). Each of these strategies has experienced limited success in producing both a humoral and cellular immune response. Overall, E2 generates a potent antibody response, but a weak CTL response in mice in contrast to the core protein (Saito et al., 1997) or nonstructural proteins (Gordon et al., 2000). One potential reason E2 has not generated a significant CTL response in previous studies is the form of the protein that is presented or encoded. Recent work by Flint et. al (Flint et al., 2000) indicates that a monomeric form of the truncated E2 glycoprotein (E2₆₆₁) preferentially binds CD81, the purported cellular receptor for HCV, as compared to the aggregated form of E2₆₆₁. Additionally, intracellular forms of E2₆₆₁ bind CD81 with greater affinity than extracellular forms.

The present invention provides RV-based vectors wherein the expression of HCV glycoproteins induce an immune response to HCV. Recombinant RV-vectors encoding HCV glycoprotein(s), or a modified version of the E2 glycoprotein with 85 amino acids of its carboxy-terminus deleted are provided herein. Additionally, recombinant RV-vectors expressing the modified version of the E2 glycoprotein along with the human CD4 transmembrane domain (TMD) and the CD4 or RV glycoprotein (G) cytoplasmic domain (CD) are provided. In addition to the RV proteins necessary for expression of immune stimulating virions, the resulting recombinant RVs stably expressed the respective HCV glycoproteins, and elicited both humoral and cellular immune responses in immunized mice.

Abbreviations

“FFU” means “foci forming units”

“MOI” means “multiplicity of infection”

“HIV” means “human immunodeficiency virus”

“SIV” means “simian immunodeficiency virus”

“HCV” means “hepatitis C virus”

“TMD” means “transmembrane domain”

“CD” means “cytoplasmic domain”

“ED” means “ectodomain”

“gag” means “group-specific antigen”

“G” means “glycprotein”

“N” means “nucleoprotein”

“L” means “Rhabdovirus polymerase”

“P” means “Rhabdovirus

“ER” means “endoplasmic reticulum”

“RV” means “rhabdovirus”

“CTL” means “cytotoxic T-lymphocyte”

“ELISA” means “enzyme-linked immunosorbant assay”

“ID” means “kilodalton”

“VLP” means “virus-like particle”

“IFN-γ” means “interferon gamma”

“PCR” means “polymerase chain reaction”

“FITC” means “fluorescence isothiocyante”

“i.p.” means “intraperitoneally”

“E:T” means “Effector:Target”

“PBMC” means “Peripheral blood mononuclear cells”

Definitions

“boost vaccine vector” is “boost virus”

“boost virus” is “boost vaccine vector”

“biological sample” refers to a sample of tissue or fluid isolated from an individual, including but not limited to, plasma, serum, spinal fluid, lymph fluid, the external sections of the skin, respiratory, intestinal, and genitourinary tracts, tears, saliva, milk, blood cells, tumors, organs, and also samples of in vitro cell culture constituents (including, but not limited to, conditioned medium resulting from the growth of cells in cell culture medium, putatively virally infected cells, recombinant cells, and cell components).

### SUMMARY OF THE INVENTION

The present invention is directed to recombinant non-segmented negative-stranded RNA virus vectors expressing an immunodeficiency virus genes as a live-viral vaccine (e.g., HIV-1 vaccine) and methods of making and using the same. More in particular the invention relates to recombinant Rhabdoviruses which express gene products of a human immunodeficiency virus and to immunogenic compositions which induce an immunological response against immunodeficiency virus infections when administered to a host. These recombinant live-viral vaccines are non-pathogenic for a wide range of animal species when administrated orally or intramuscularly and induce protective immune responses such as neutralizing antibody response and long lasting cellular (such as cytotoxic T lymphocyte (CTL)) responses against the immunodeficiency viruses.

In general aspects, the invention is a recombinant non-segmented negative-stranded RNA virus vector having: (a) a modified negative-stranded RNA virus genome that is modified to have one or more new restriction sites, or not to have one or more genes otherwise present in the genome; (b) a new transcription unit that is inserted into the modified negative-stranded RNA virus genome to express heterologous nucleic acid sequences; and (c) a heterologous viral nucleic acid sequence that is inserted into the new transcription unit, where the recombinant non-segmented negative-stranded RNA virus vector is replication competent, and the heterologous viral nucleic acid sequence encodes an antigenic polypeptide.

Specifically, in one embodiment of the invention, the recombinant non-segmented negative-stranded RNA virus vector that is used as a live-viral vaccine is a recombinant Rhabdovirus vector. This vector includes (a) a modified Rhabdovirus genome; (b) a new transcription unit inserted into the Rhabdovirus genome to express heterologous nucleic acid sequences; and (c) a heterologous viral nucleic acid sequence that is inserted into the new transcription unit, where the recombinant Rhabdovirus vector is replication competent, and the heterologous viral nucleic acid sequence encodes an antigenic polypeptide. The modified Rhabdovirus genome is, for example, modified rabies virus genome or a modified vesicular stomatitis virus genome. The modifications in the Rhabdovirus genome include creation of new restriction sites and/or deletion of one or more genes such as the native G (glycoprotein) gene of the Rhabdovirus, ψ gene of rabies virus, etc. In some instances, the modified Rhabdovirus genome has a further modification to have a glycoprotein from another class of virus in place of the native glycoprotein. The glycoprotein from another class of virus is vesicular stomatitis virus glycoprotein. In some other instances, the modified rabies virus genome has a third modification to have contiguity of structural genes different from that in the rhabodvirus genome after the second modification.

The term heterologous viral nucleic acid as used herein refers to the viral nucleic acid that encodes the antigenic polypeptide that induces immune response. For example, a full-length HIV envelope protein, HIV gp160, HIV gag, HIV gp120, and full-length SIV envelope protein are some of the antigenic polypeptides that are expressed in the recombinant viral vectors of the present invention. The term heterologous viral nucleic acid as used herein does not include the native gene sequences of the one or more classes of Rhabdoviruses in a recombinant Rhabdovirus such as, for example, VSV G gene in the recombinant RV.

In the case of a modified Rhabdovirus genome where G gene is deleted, the sequence of the cytoplasmic domain of Rhabdovirus G gene is fused to other sequences before cloning into the modified Rhabdovirus genome. One such example is a chimeric VSV/RV glycoprotein where the fusion protein has VSV ectodomain and transmembrane domain, and RV cytoplasmic domain. Another such example is a chimeric HIV-1/RV glycoprotein where the fusion protein has HIV-1 gp160 ectodomain and transmembrane domain, and RV cytoplasmic domain. Thus, in some cases, the heterologous viral nucleic acid is fused to the sequence of the cytoplasmic domain of the G gene of the modified Rhabdovirus genome to produce a chimeric protein such that the resulting chimeric protein has a fusion between the transmembrane domain of the heterologous protein and cytoplasmic domain of the glycoprotein. In some cases, the glycoprotein gene of the recombinant Rhabdovirus is deleted and the heterologous viral nucleic acid is fused to the sequence of the cytoplasmic domain of the G gene of the modified Rhabdovirus genome to produce a chimeric protein which functionally substitutes for the recombinant Rhabdoviruses glycoprotein gene.

In another embodiment of the invention a recombinant Rhabdovirus that expresses a functional HIV envelope protein is provided. The recombinant Rhabdovirus is replication-competent. The Rhabdovirus can be a recombinant rabies virus or a recombinant vesicular stomatitis virus.

The HIV envelope protein expressed from the recombinant Rhabdovirus is from any HIV-1 isolate.

In still another embodiment of the invention, a recombinant Ψ gene deficient Rhabdovirus having a heterologous nucleic acid segment encoding an immunodeficiency virus envelope protein or a subunit thereof is provided. In such cases, the recombinant Ψ gene deficient Rhabdovirus is a rabies virus and the immunodeficiency virus envelope protein, or a subunit thereof, is from a human immunodeficiency virus or from a simian immunodeficiency virus. The subunit or a fragment of the immunodeficiency envelope protein includes fragments having only a part of the contiguous amino acids of the envelope protein. These subunits or fragments include, for example, HIV gp120, HIV gp41, HIV gp40, the envelop proteins expressed by HIV_{NL4-3} and HIV_{89.6,} and the subunits of other immunodeficiency viruses.

In yet another embodiment of the invention, a method of inducing an immunological response in a mammal is provided. This method includes the steps of: (a) delivering to a tissue of the mammal a recombinant Rhabdovirus vector that expresses a functional immunodeficiency virus envelope protein, or a subunit thereof, effective to induce an immunological response to the envelope protein; (b) expressing the envelope protein, or the subunit thereof, in vivo; (c) boosting the animal by delivering an effective dose of an isolated immunodeficiency virus envelope protein, or a subunit thereof, in an adjuvant or by delivering an effective dose of a boost vaccine vector; and (d) inducing a neutralizing antibody response and/or long lasting cellular immune response thereto to protect the mammal from an immunodeficiency virus.

The recombinant Rhabdovirus has a rabies virus genome. In the method where the rabies virus genome is used, it is deficient in T gene. In some cases, rabies virus genome is also deficient in a rabies virus glycoprotein gene or rabies virus genome has glycoprotein gene from another class of Rhabdovirus in place of the rabies virus glycoprotein. Boosting the animal can be done by delivering an effective dose of a boost vaccine vector instead of the isolated immunodeficiency virus envelope protein.

In another embodiment of the invention an immunogenic composition having any of the above mentioned recombinant Rabdoviruses along with an adjuvant is provided.

In yet another embodiment of the invention a method of inducing an immunological response in a mammal is provided which includes the steps of: (a) delivering to a tissue of the mammal a non-segmented negative-stranded RNA virus that expresses a functional immunodeficiency virus envelope protein, or a subunit thereof, effective to induce an immunological response to the envelope protein; (b) expressing the envelope protein, or the subunit thereof, in vivo; (c) boosting the animal by delivering an effective dose of an isolated immunodeficiency virus envelope protein, or a subunit thereof, in an adjuvant or by delivering an effective dose of a boost vaccine vector; and (d) inducing a neutralizing antibody response and/or long lasting cellular immune response thereto to protect the mammal from an immunodeficiency virus.

The method where the non-segmented negative-stranded RNA virus is used includes a Rabies virus or a Vesicular Stomatitis virus.

It is a further object of the invention to present a method of treating a mammal infected with an immunodeficiency virus. A non-segmented negative-stranded RNA virus that expresses a functional immunodeficiency virus envelope protein, or subunit thereof is administered to the mammal. This RNA virus will express the functional immunodeficiency virus envelope protein, or subunit thereof. An effective dose of an isolated immunodeficiency virus envelope protein, or subunit thereof, in an adjuvant or an effective dose of a boost vaccine vector is delivered to the mammal, thereby inducing a neutralizing antibody response and/or long lasting cellular immune response to the functional immunodeficiency virus envelope protein, or subunit thereof. In one embodiment the immunodeficiency virus is any HIV-1 virus. In another embodiment the non-segmented negative-stranded RNA virus is a Rhabdovirus. In a further embodiment there is an induction of mucosal immunity to the functional immunodeficiency virus envelope protein, or subunit thereof. In another embodiment the long-lasting cellular response is a cross-reactive CTL response wherein the cross-reactive CTLs are directed against envelope proteins, or subunits thereof, from different immunodeficiency virus strains.

It is another object of the invention to present a method of protecting a mammal from an immunodeficiency virus infection. A non-segmented negative-stranded RNA virus that expresses a functional immunodeficiency virus envelope protein, or subunit thereof is administered to the mammal. This RNA virus will express the functional immunodeficiency virus envelope protein, or subunit thereof, thereby thereby inducing a neutralizing antibody response and/or long lasting cellular immune response to the functional immunodeficiency virus envelope protein, or subunit thereof. In one embodiment the immunodeficiency virus is any HIV-1 virus. In another embodiment the non-segmented negative-stranded RNA virus is a Rhabdovirus. In a further embodiment there is an induction of mucosal immunity to the functional immunodeficiency virus envelope protein, or subunit thereof. In another embodiment the long-lasting CTL response is a cross-reactive CTL response wherein the cross-reactive CTLs are directed against envelope proteins, or subunits thereof, from different immunodeficiency virus strains.

### BRIEF DESCRIPTION OF THE FIGURES

FIG. 1. Schematically shows a method for the construction of recombinant RV genomes.

FIG. 2. A graph showing One-step growth curves of BSR cells that were infected with the recombinant RVs (SBN, SBN-89.6, and SBN-NL4-3)

FIG. 3. Western blot analysis of recombinant rabies viruses (RVs) expressing HIV-1 gp160.

FIG. 4. A composite photograph showing Sup-Ti cells after these cells were infected (using a MOI of 1) with SBN, SBN-89.6, or SBN-NL4-3.

FIG. 5. A graph showing ELISA reactivity of mouse sera against HIV-1 gp120.

FIG. 6. Western blot analysis of mice serum antibody response to HIV-1 antigens.

FIG. 7. Schematic representation of a method for the construction of RV-based expression vectors with foreign viral glycoproteins.

FIG. 8. Schematic representation of a method for the construction of full-length and RV-glycoprotein deleted RVs expressing HIV-1 gp160.

FIG. 9. CTLs from HIV-1 gp160 immunized mice induce long-lasting HIV-1 gp160-specific CTLs. Groups of three 6- to 8-week-old female BALB/c mice (Harlan Sprague) are inoculated i.p. with 2×10⁷ foci-forming units of recombinant RV expressing HIV-_{INL4-3} envelope protein. 105 to 135 days after the single inoculation, spleens are aseptically removed and single cells suspensions are prepared (infra). Stimulator cells are prepared (infra), then added back to the effector cell population at a ratio of 3:1. Cytolytic activity of cultured CTLs is determined by measurement of the percent ⁵¹Cr released (infra).

FIG. 10. CTLs from HIV-1 gp160 immunized mice cross-kill target cells expressing heterologous HIV-1 envelope proteins. Groups of six 6- to 8-week-old female BALB/c mice are inoculated i.p. with 2×10⁷ foci-forming units recombinant RV expressing HIV-1 envelope protein from strains NL4-3 (A) or 89.6 (B). Three and four weeks after the single inoculation, spleens were aseptically removed and splenocytes were stimulated in-vitro with vaccinia virus expressing the homologous HIV-1 envelope protein (infra). Target cells are prepared by infection with vaccinia virus expressing HIV-1 envelope proteins from strains NL4-3 (vCB41), 89.6 (vBD3), JR-FL (vCB28), or Ba-L (vCB43). Chromium release assays are completed (infra). The results are shown from two different, independent experiments.

FIG. 11. Cytolytic activity is mediated by CD8+T-cells. Groups of three 6- to 8-week-old female BALB/c mice are inoculated i.p. with 2×10 foci-forming units recombinant RV expressing HIV-1 envelope protein from the NL4-3 strain. Eighteen weeks after the single inoculation, spleens are aseptically removed and splenocytes are stimulated in vitro with vaccinia virus expressing HIV-I_{NL4-3} envelope protein (infra). Seven days post in vitro stimulation, CD8⁺ T-cells are depleted from the cell culture (CD8⁻) and enriched (CD8⁺) using Dynabeads Mouse CD8 (Lyt2), as described by the manufacturer. Chromium release assays are completed (infra) on cultures depleted (CD8⁻) or enriched (CD8⁺) of CD8 T-cells, or unprocessed cultures (CD8⁺/CD8⁻). Target cells are prepared (infra) by infection with vaccinia virus expressing HIV-1 envelope proteins from NL4-3 (vCB41). Background levels were equal to, or below, 6% specific lysis.

FIG. 12. Construction of recombinant RV genomes. At the top (A), the SPBN vector derived from the RV vaccine strain SAD B16 is illustrated. Through site directed mutagenesis and a PCR strategy, a transcription Stop/Start signal was introduced in addition to four unique restriction enzyme sites (SmaI, Pacl, BsiWI and NheI). The HCV proteins (blue box) were introduced into pSPBN using the BsiWI and NheI sites resulting in the plasmids pSBPN-E1E2p7 (B), pSPBN-E2CD4 (C), and pSPBN-E2CD4G (D). E2CD4 and E2CD4G are a truncated version of HCV E2 lacking 85 amino acids at their C-terminus, fused to the TMD (green box) and CD of human CD4 (light blue box) or TMD of CD4 and CD of RV G (red box), respectively. FIG. 13. Immunoflourescence studies of recombinant RVs expressing HCV proteins. BSR cells were infected with the recombinant RVs SBPN (A, A′, A″), SPBN-E1E2p7 (B, B′, B″), or pSPBN-E2CD4G (C, C′, C″) at a MOI of 0.1 and 48 hours after infection, cell were fixed, permeabilized (A′, B′, C′, A″, B″, C″) or not (A, B, and C), and stained with a monoclonal antibody directed against E2 (A, A′, B, B′, C, and C′) or RV N (A″, B″, or C″).

FIG. 14. Western Blot analysis of HCV proteins expressed by RV. BSR cells were infected with recombinant RVs as indicated (SPBN, SPBN-EIE2p7, SPBN-E2CD4, SPBN-E2CD4G at a MOI of 5. Cell lysates were separated by SDS-PAGE and transferred to a nitrocellulose membrane. Blots were probed with monoclonal antibodies directed against the HCV E1 and E2 glycoproteins as indicated (A-E1, UE1+E2 or OCE1).

FIG. 15. Incorporation of HCV proteins in recombinant RVs. Purified particles of SPBN, SPBN-E2CD4 or SPBN-E2CD4G were separated by SDS-PAGE and visualized by Coomassie blue staining (CB, lanes 1, 2, 3) or transferred to a nitrocellulose membrane before ((X-E2) or after digestion with N-glycosidase F ((X-RV-G-tail). Blots were probed with a monoclonal antibodies directed against the HCV E2 ((X-E2 lanes 4, 5 and 6) or a polyclonal rabbit serum specific for the RV G CD ((X-RV-G-tail, lanes 7, 8, 9).

FIG. 16. Recombinant SPBN-E2CD4G virions as a diagnostic tool. ELISA plates were coated with recombinant HCV E2 derived from purified SPBN-E2CD4G virions and incubated with sera from three HCV-positive patients (HCV1-3), pooled sera from HCV and RV-negative donors (HCV−/RV−). Sera from a RV-vaccinated donor (HCV−/RV+) and HIV-1-positive patient (HIV+/RV−) served as controls. The error bars indicate the standard deviations.

FIG. 17. ELISA reactivity of mouse sera against HCV E2. Four groups of five mice each were immunized with live recombinant RV (SPBN, SPBN-E2CD4G) as indicated, and 5 weeks after the initial immunization the mice were boosted twice with killed SPBN-E2CD4 or SPBN virions as indicated in the Figure (Boost). Ten days after the second boost, sera were collected and analyzed by ELISA. Each bar represent the reactivity of a single mouse serum at a 1:100 serum dilution.

FIG. 18. Immunization of mice with SPBN-E1E2p7 induces HCV E2-specific CTLs. 6-8 week old female BALB/c mice were immunized intraperitonially (i.p.) with 1×10 FFU of SPBN-E11E2p7. Spleens were harvested 11 weeks after immunization, cultured and stimulated with the E2 peptide 1323 and IL-2. A standard chromium release assay was performed one week after harvesting, against P815 cells pulsed (+peptide) or not (−peptide) with peptide 1323.

FIG. 19. Construction of recombinant RVs and expression of HIV-1 Gag from RV. (A) The RV vaccine strain-based expression vector is shown at the top of the figure (SPBN). The HIV-1_{NL4-3} gag coding region was amplified by PCR and cloned into SPBN using the BsiWI and NheI sites. The resulting plasmid was designated pSPBN. Expression of HIV-1 Gag from recombinant SPBN-Gag (B). HeLa cells were infected with a MOI of 0.1 with SPBN (panel A and A′) or SPBN-Gag (panel B and B′), and analyzed by immunofluorecence microscopy with an antibody directed against RV N (panel A and B) or HIV-1 Gag (panel A′ and B″) 48 hours after infection.

FIG. 20. Western blot analysis of recombinant RVs expressing HIV-1 Gag. HeLa cells were infected with a MOI of 5 with SPBN or SPBN-Gag and lysed 24 hours later. Proteins were separated by SDS-PAGE and analyzed by Western blotting. An antibody directed against HIV-1 p24 antigen detected a prominent band at the expected size for HIV-1 (lane 2). No signal was detected for SPBN infected control cells (lane 1). Lysates from HIV-1_{NL4-3} infected SupT1 cells served as a control (lane 3). An antibody directed against RV G confirmed the infection of the cells with RV (lanes 4, 6).

FIG. 21. Evaluation of Recombinant RV Expressing HIV-1 Gag by EM. HeLa cells were infected with SPBN-Gag with a MOI of 1 for 48 hours. Cells were fixed at room temperature in neutral buffered 2.5% glutaraldehyde and gelled into warm agar. The cells were post-fixed in 1% OsO₄, dehydrated in graded ethanol and propylene oxide, and embedded in Spurr's epoxy. Thin sections were cut and stained with uranly acetate and lead citrate and examined with a LEO EM10 electron microscope at 60 kV. The figure shows large numbers of bullet shaped RV particles (A, white arrows) and late budding and immature HIV-1 particles (A, black arrows) both on the plasma membrane and in cytoplasmic vacuoles (Magnification: 43,000[A] and 131,000 [B-D]).

FIG. 22. HIV-1-specific CTLs after a single immunization with SPBN-Gag. Balb/c mice were inoculated i.p. with 10⁷ foci-forming units of recombinant RV expressing HIV-1 Gag. Three weeks post-immunization, splenocytes from three mice were pooled and stimulated with p24 peptide (AMQMLKETI; SEQ. ID. NO: 18). Cytolytic activity of cultured CTLs was measured after 7 days. The target cells (P815) were pulsed or not with p24 peptide.

FIG. 23. Induction of HIV-1 Gag-specific IFN-γ-producing CD8⁺ T-cells in BALB/c mice after a single immunization with SPBN-Gag. Groups of 6- to 8-week-30 old female Balb/c mice were inoculated i.p. with 10⁷ foci-forming units of recombinant RV expressing HIV-1 Gag (SPBN-Gag) or vector alone (SPBN). Nine weeks post-immunization, mice were challenged with recombinant vaccinia virus expressing HIV-1 Gag (vv-Gag) or the HCV structural protein (vv-HCV). Five days later, spleens were removed and cells were stimulated in-vitro with, or without, p24 peptide (AMQMLKETI; SEQ. ID. NO: 18) for 16 hours as described in Material and Methods. Cells were stained with PE-conjugated monoclonal rat anti-mouse CD8a antibody and a FITC-conjugated rat anti-mouse IFN-γ. The number in each panel indicates the percentage of CD8+ T-cells secreting IFN-γ.

FIG. 24. Staining of PBMCs or splenocytes with the K^{d}-AMQMLKETI (SEQ. ID. NO: 18) MHC-peptide tetrameric complex. Mice were immunized as described in FIG. 23 and eleven weeks post-immunization mice were challenged with recombinant vaccinia virus expressing the HCV structural protein (A and B) or HIV-1 Gag (A′ and B′). Five days later, PBMC (A, A′) or splenocytes (B, B′) were isolated and cells were stained with FITC-conjugated rat anti-mouse CD8 antibody and the K^{d}-AMQMLKETI MHC-peptide tetrameric complex.

### DETAILED DESCRIPTION OF THE INVENTION

Rhabdoviruses such as Rabies virus and Vesicular Stomatitis virus are members of the family Rhabdoviridae. Rabies virus possesses a negative stranded RNA genome of approximately 12 kb. The genome is modularly organized and similar to that of vesicular stomatitis virus (VSV). These Rhabdoviruses encode five structural proteins. The five open reading frames coding for the viral structural proteins are nucleoprotein (N), phosphoprotein (P), matrix protein (M), glycoprotein (G), and polymerase (L). After infection, the viral polymerase-complex (P and L) begins transcription at the 3′ end of the encapsidated genome to generate a short leader RNA followed by sequential synthesis of five viral RNAs. The nucleoprotein (N), the phosphoprotein (P), the viral polymerase (L), and the genomic RNA form a helical ribonucleoprotein complex (RNP). The RNP is surrounded by a host cell-derived envelope membrane which contains the matric protein (M) on the inner side of the membrane, and the transmembrane glycoprotein (G) which mediates binding of the virus to specific receptors on the cell membrane.

The generation of non-segmented negative-strand RNA viruses entirely from cDNA has been reported by the inventors. (Schnell et. al., EMBO, 13:4195-4203, 1994). The approach involved intracellular expression of anti-genomic RNA in cells also expressing the viral proteins required for formation of an active RNP complex, namely, the nucleoprotein (N), the phosphoprotein (P), and the viral polymerase (L). This method avoids problems of antisense that are encountered when expressing the non-encapsidated negative-strand genomic RNAs, and positive strand mRNAs, and the same method was later also successful in the recovery of another Rhabdovirus, VSV. (Lawson et al., PNAS, USA, 92:4477-81, 1995).

In the present invention a number of recombinant Rhabdovirus vectors are generated and are used to express functional genes, including, but not limited to, full-length HIV-1 envelope proteins and HCV envelope proteins. From the recombinant Rhabdovirus vectors of the invention all the dominant epitopes for neutralizing antibodies, cytotoxic T-lymphocytes (CTL), and antibody-dependent cell cytotoxicity are expressed at one time. The construction of different recombinant Rhabdovirus vectors expressing HIV, HCV, SIV or other viral genes is described in the following paragraphs.

Recombinant Rhabdovirus Expression Vectors

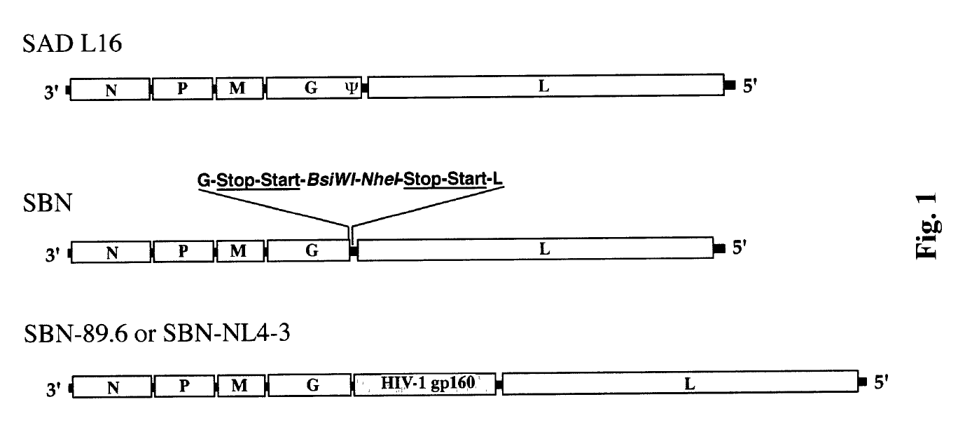

Several different recombinant Rhabdovirus-based and replication-competent expression vectors that express heterologous genes or gene sequences are constructed. In one aspect of the invention an expression vector with its own glycoprotein is constructed. The genome of this recombinant expression vector can be represented as: 3′-N-P-M-G-X-L-5′ where X=foreign gene (e.g. HIV-1 gp160, HIV-1 gag, or any other HIV-1 gene; any SIV, HIV-2, Hepatitis C gene, or any other viral antigen) (see FIG. 1). X can be cloned at different genome sites to regulate expression levels.

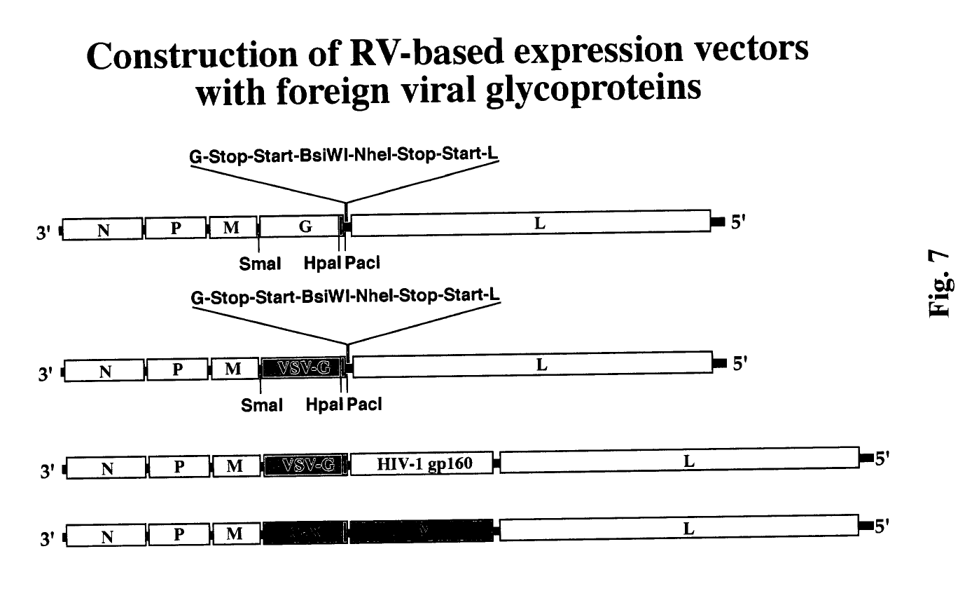

In another aspect of the invention an expression vector with a glycoprotein from another virus or another viral serotype is constructed (see FIG. 7 as an example for the RV vector with VSV glycoprotein). This vector is used as boost virus to induce a stronger immune response. The genome of this recombinant expression vector is represented as: 3′-N-P-M-G (from another virus or viral serotype)-X-L-5′ (for example, 3′-N-P-M-G from VSV serotype Indiana)-X-L-₅) where X=foreign gene specific (e.g. HIV-1 gp160, HIV-1 gag, or any other HIV-1 gene; any SIV or HIV-2 gene, HCV, HCV E2, or any other viral antigen). X can be cloned at different genome sites to regulate expression levels. The present invention relates to constructs of recombinant RVs (rabies viruses) expressing HIV-1 gp 160, where the RV glycoprotein (G) is replaced with that of a chimeric vesicular stomatitis virus (VSV) G/RV-cytoplasmic domain (serotype Indiana or New Jersey). Of note, this method is not restricted to VSV glycoprotein. Because Rhabdoviruses have only a single surface protein on their virions, chimeric RV/VSV viruses are not neutralized by the humoral response against the RV G and therefore allow a second productive infection. The use of a recombinant chimeric RV/VSV can be used to display the properly folded HIV-1 envelope protein on the surface of the infected cell.

The present invention further relates to constructs of recombinant RVs containing the gene encoding the ectodomain of HCV E2, with the 85 carboxy-terminal amino acids deleted, fused to the transmemebrane domain (TMD) and cytoplasmic domain (CD) of human CD4, or the TMD of CD4 and the CD of RV G.

It should be noted that repeated expression of the RV nucleoprotein, which was previously shown to be an exogenous superantigen (Lafon, et al., Nature, 358, 507-10, 1992; Lafon, M. Research in Immunology, 144:209-13, 1993), might help to enhance the immune response against the HIV-1 envelope or matrix and nucleocapsid proteins, as well as the HCV E2 envelope. In case of Rabies Virus (RV) the cytoplasmic domain of the RV glycoprotein is fused to the foreign glycoproteins.

It should be noted that all genes within the recombinant genome can be rearranged to attenuate the virus or to enhance transcription of the foreign gene. For example, a recombinant RV with rearranged genome, VSV glycoprotein, and HIV-1 gp160 (X) can be constructed to have: 3′-X-N-P-G(VSV serotype NJ)-M-L-5′.

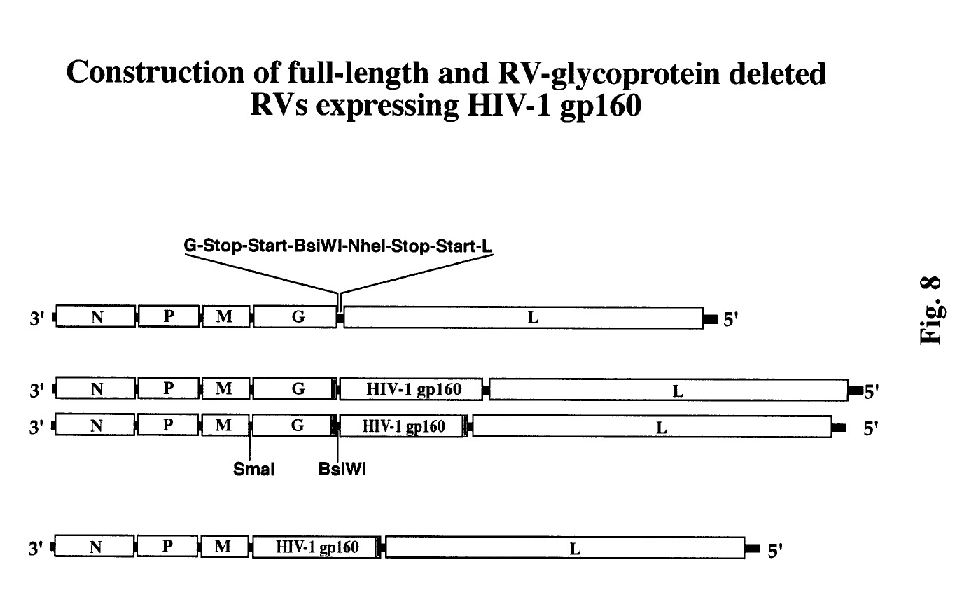

In still another aspect of the invention a recombinant expression vector (either RVs or VSVs) having a foreign glycoprotein instead of their own is constructed for entry into specific host cells, i.e., to mimic the tropism of another virus (e.g., HIV-1, Hepatitis C) in order to induce a stronger immune response (FIG. 8). This construct can be represented as 3′-N-P-M-HIV-1-gp160-L. Alternatively these constructs can have, in addition, their own glycoproteins (e.g., 3′-N-P-M-HIV-1-gp160-G-L). Again, it should be noted that all genes within the recombinant genome can be rearranged to attenuate the virus or to enhance transcription of the foreign gene. Transgenic mice expressing human CD4 and CXCR4 are generated to analyze the in vivo induction of an immune response of the G-related RVs expressing HIV-1 gp160/RVG and HCV E2.

In still another aspect of the invention a recombinant expression vector (either RV or VSV) having multiple genes and multiple transcription stop/start signals is constructed. This construct is represented as 3′-N-P-M-G-X-Y-L-5′where X and Y are heterologous genes. For example, X can be HIV-1 gp160 and Y can be HIV-1 gag or X can be HCV E1 and Y can be HCV E2. An alternative construct can be 3′-N-Z-P-M-G-X-Y-L-5′ where, for example, X can be HIV-1 gp160, Y can be HIV-1 gag and Z can be HIV-1 tat; or X can be HCV E1, Y can be HCV E2, and Z can be HCV p7.

A Rhabdovirus Vaccine

In a preferred embodiment, an immunodeficiency virus vaccine based on recombinant rabies virus vectors is described. Rabies virus (RV) is a negative-stranded RNA virus of the Rhabdovirus family and it possesses a relatively simple, modular genome organization coding for five structural proteins (supra and Conzelmann, et al., Virology, 175:485-99, 1990). The present invention relates to an RV vaccine strain-based vector, which is non-pathogenic for a wide range of animal species when administrated orally or intramuscularly. This vector shows advantages over other viral vectors, for several reasons. First, its modular genome organization makes genetic modification easier than for the majority of more complex genomes of DNA and plus-stranded RNA viruses. Second, Rhabdoviruses have a cytoplasmic replication cycle and there is no evidence for recombination and/or integration into the host cell genome. (Rose, et al., Rhabdovirus genomes and their products, Plenum Publishing Corp., New York, 1997). In contrast to most other viral vectors only a negligible seropositivity exists in the human population to RV and immunization with a RV-based vector against HIV-1 (or HCV, infra) will not interfere with immunity against the vector itself. In addition, RV grows to high titers 10⁹ foci forming units (FFU) in various cell-lines without killing the cells, which probably results in longer expression of HIV-1 genes (or HCV genes, infra) compared to a cytopathogenic vector.

Generation of Recombinant Vectors

The following different recombinant rabies virus vectors are constructed. A new infectious Rabies Virus (RV) vector with a deletion of the ψ-gene (a ˜400 bases long non-coding sequence fused to the G RNA) and new transcription unit containing a short transcription Stop/Start signal (to express foreign genes) and two single sites (BsiWI and NheI) to introduce foreign genes is constructed. This vector also contains a SmaI site upstream of the RV glycoprotein, which is used to delete the RV glycoprotein gene (G). The vector is called RV-SBN. RVs expressing HIV-1gp160 ecto- and transmembrane domain fused to the RV G cytoplasmic domain (HIV-1gp160-RVG) are constructed. The chimeric gp160/RVG protein is expressed by RV and incorporated into RV virions. RVs expressing HCV glycoproteins are also generated (SPBN-E1E2p7, infra). Additionally, two similar RV recombinant viruses are also generated. SPBN-E2CD4 (infra) contains the ectodomain of HCV E2, with a 85 amino acid deletion at the carboxy-terminus, fused to the trans- and cytoplasmic domains of human CD4. Alternatively, the ectodomain of HCV E2 is fused to the transmembrane domain of human CD4 and the cytoplasmic domain of RV G (SPBN-E2CD4G, infra). A recombinant virus displaying a foreign envelope protein on its surface will induce a strong immune response against this antigen.

Another RV vector is also generated which is identical to RV-SBN but has, in addition, a single PacI site downstream of RV G protein. This vector is used to functionally replace RV G with VSV G or other viral glycoproteins. This vector is called RV-SPBN and is used as a boost vaccine vector or a boost virus.

As shown in FIG. 7, a recombinant rabies virus based expression vector with foreign viral glycoproteins is constructed and the recombinant virus is recovered. For this construct a SmaI restriction enzyme site is introduced downstream of the M/G transcription Stop/Start sequence and a PacI site upstream of the synthetic transcription Stop/Start sequence, which is used to express foreign genes from the RV vector. These two sites (SmaI/Pac) can be used to replace the RV glycoprotein with that from other viruses. In FIG. 7 a chimeric VSV/RV glycoprotein (VSV ectodomain and transmembrane domain, RV cytoplasmic domain), in combination with HIV-1 is shown as an example. However, it should be noted that this method can be applied to every glycoprotein and foreign antigen in different Rhabdoviruses (see infra), as shown in the same figure (glycoprotein X, foreign protein Y).

In another experiment, recombinant RVs expressing chimeric gp160/RV G without expressing RV G (G-deleted RVs) are generated. These G-deleted RVs have a different tropism as compared to wild-type RV (which infects most cells) and specifically infect only cells expressing the HIV-1 receptor human CD4 and one of the HIV-1 coreceptors (eg, CXCR4 or CCR5).

Both the full-length and RV-glycoprotein deleted recombinant rabies RVs are constructed and recovered (FIG. 8). The Smal and BsiWI restriction enzyme sites are used to delete RV glycoprotein and fuse the M/G transcription Stop/Start sequence to the HIV-1/RV chimeric glycoprotein (HIV-1 gp160 ectodomain and transmembrane domain, RV cytoplasmic domain). The recovered RV-vector is, analogous to the HIV-1 virus, specific for cells expressing human CD4 and the appropriate HIV-1 co-receptor. It should be noted that this method can be applied to every glycoprotein which supports infection of certain cell types by rhabdoviruses. It can also be used to express additional foreign antigens (HIV-1 Gag, HIV protease, SIV proteins, Hepatitis A, B or C proteins (see HCV, infra), and other viral and non-viral proteins).

In still another aspect of the invention a recombinant replication-competent rabies virus expression vector having all of the above combinations can be constructed. For example, a recombinant rabies virus vector having other glycoproteins (especially to construct boost viruses) without or with their own G, having genome rearrangements, and expressing multiple viral antigens from the same or different viruses (e.g. HIV-1 gp160, Hepatitis B, Hepatitis C (infra)).

Product, s Methods and Compositions

There are provided by the invention, products, compositions and methods for assessing treating viral diseases, particularly HIV (AIDS) and HCV (hepatitis) and administering a recombinant Rhadovirus of the invention to an organism to raise an immunological response against invading viruses, especially against immunodeficiency virus infections and hepatitis C virus infections.

Methods for Induction of an Immune Response

Another aspect of the invention relates to a method for inducing an immunological response in an individual, particularly a mammal, which involves inoculating the individual with a recombinant virus of the invention followed by the appropriate recombinant protein boost, adequate to produce antibody and/or T cell immune response to protect the individual from infection, particularly immunodeficiency infection and hepatitis C infection, and most particularly HIV-1 and 2 infections, as well as HCV infections. Also provided are methods whereby such immunological response slows the HIV replication and the HCV replication.

Yet another aspect of the invention relates to a method of inducing immunological responses in an individual which comprises delivering to such individual a nucleic acid vector, sequence or ribozyme to direct the expression of HIV envelope polypeptides (or HCV envelope polypeptides, or a fragment or a variant thereof, infra), or a fragment or a variant thereof, for expressing the HIV envelope polypeptide (or HCV envelope polypeptides, or a fragment or a variant thereof, infra), or a fragment or a variant thereof, in vivo in order to induce an immunological response, such as, to produce antibody and/or T cell immune response. Antibody and/or T cell responses include, for example, cytokine-producing T cells or cytotoxic T cells, to protect the individual, preferably a human, from the viral disease, whether that disease is already established within the individual or not. One example of administering the gene is by accelerating it into the desired cells as a coating on particles or otherwise. Such nucleic acid vector may comprise DNA, RNA, a ribozyme, a modified nucleic acid, a DNA/RNA hybrid, a DNA-protein complex or an RNA-protein complex.

Compositions that Induce an Immunological Response

A further aspect of the invention relates to an immunological composition that when introduced into an individual, preferably a human, capable of having induced within it an immunological response. The immunological response that is induced is to a polynucleotide and/or polypeptide encoded therefrom, wherein the composition comprises a recombinant Rhabdoviruses of the invention which encodes and expresses an antigen of an exogeneous viral protein, such as HIV envelope protein or polypeptide, HCV envelope protein or peptide, etc. Specifically, the exogeneous polypeptides include antigenic or immunologic polypeptides. The immunological response is used therapeutically or prophylactically and takes the form of antibody immunity and/or cellular immunity, such as cellular immunity arising from CTL or CD4+ T cells.

In a further aspect of the invention there are provided compositions comprising a Rhabdovirus vector of the present invention for administration to a cell or to a multicellular organism.

Pharmaceutical Compositions

The Rhabdovirus vectors of the invention may be employed in combination with a non-sterile or sterile carrier or carriers for use with cells, tissues or organisms, such as a pharmaceutical carrier suitable for administration to an individual. Such compositions comprise, for instance, a media additive or a therapeutically effective amount of a recombinant virus of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

The recombinant vectors of the invention may be employed alone or in conjunction with other compounds, such as therapeutic compounds.

Methods of Administration

The pharmaceutical compositions may be administered in any effective, convenient manner including, for instance, administration by intravenous, intraperitoneal, intramuscular, subcutaneous, intranasal or intradermal routes among others. In therapy or as a prophylactic, the active agent may be administered to an individual as an injectable composition, for example as a sterile aqueous dispersion, preferably isotonic. The pharmaceutical compositions of the invention are preferably a systematic effect against relevant viral administered by injection to achieve pathogens.

For administration to mammals, and particularly humans, it is expected that the daily dosage level of the active composition of the invention will be from 10² FFU to 10⁸ FFU of virus in the composition or 10 μg/kg to 10 mg/kg of body weight of recombinant protein. The physician in any event will determine the actual dosage and duration of treatment that will be most suitable for an individual and can vary with the age, weight and response of the particular individual. The above dosages are exemplary of the average case. There can, of course, be individual instances where higher or lower dosage ranges are merited, and such are within the scope of this invention.

A vaccine composition is conveniently in injectable form. Conventional adjuvants may be employed to enhance the immune response. A suitable unit dose for vaccination is preferably administered daily and with or without an interval of at least 1 week. With the indicated dose range, no adverse toxicological effects are observed with the compounds of the invention that would preclude their administration to suitable individuals.

Immunoassay and Diagnostic Kits

The recombinant virions of the present invention are useful for producing an HCV antigenic polypeptide(s), for example the E2 glycoprotein, or subunits thereof, which reacts immunologically with a biological sample from a patient, such as, but not limited to, serum, containing HCV antibodies. The present invention also encompasses antibodies raised against the HCV specific epitopes in these antigenic polypeptides, which are useful in immunoassays to detect the presence of the HCV virus and/or viral antigens, in biological samples. Design of the immunoassays is subject to a great deal of variation, and many formats are known in the art. The immunoassay will utilize at least one viral epitope derived from HCV. In one embodiment, the immunoassay uses a combination of viral epitopes derived from HCV. These epitopes may be derived from the same, for example from the E2 glycoprotein, or from different viral polypeptides, for example from the E2 and E1 polypeptides. An immunoassay may use, for example, a monoclonal antibody directed towards a viral epitope(s), a combination of monoclonal antibodies directed towards epitopes of one viral antigen, monoclonal antibodies directed towards epitopes of different viral antigens, polyclonal antibodies directed towards the same viral antigen, or polyclonal antibodies directed towards different viral antigens.

Protocols may be based, for example, upon competition, or direct reaction, or sandwich type assays (infra). Protocols may also, for example, use solid supports, or may be by immunoprecipitation. Most assays involve the use of labeled antibody or polypeptide; the labels may be, for example, enzymatic, fluorescent, chemiluminescent, radioactive, or dye molecules. Assays that amplify the signals from the probe are also known; examples of which are assays which utilize biotin and avidin, and enzyme-labeled and mediated immunoassays, such as ELISA assays (infra).

Immunoassaying for Anti-HCV Antibody(s)

Typically, an immunoassay for an anti-HCV antibody(s) will involve selecting and preparing the test sample suspected of containing the antibodies, such as a biological sample, then incubating it with an antigenic (i.e., epitope-containing) HCV polypeptide(s) under conditions that allow antigen-antibody complexes to form, and then detecting the formation of such complexes. Suitable incubation conditions are well known in the art. The immunoassay may be, without limitations, in a heterogeneous or in a homogeneous format, and of a standard or competitive type.

In a heterogeneous format, the polypeptide is typically bound to a solid support to facilitate separation of the sample from the polypeptide after incubation. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride, diazotized paper, nylon membranes, activated beads, and Protein A beads. The solid support containing the antigenic polypeptide is typically washed after separating it from the test sample, and prior to detection of bound antibodies. Both standard and competitive formats are known in the art.

In a homogeneous format, the test sample is incubated with antigen in solution. For example, it may be under conditions that will precipitate any antigen-antibody complexes which are formed. Both standard and competitive formats for these assays are known in the art.

In a standard format, the amount of HCV antibodies forming the antibody-antigen complex is directly monitored. This may be accomplished by determining whether labeled anti-xenogenic (e.g., anti-human) antibodies which recognize an epitope on anti-HCV antibodies will bind due to complex formation. In a competitive format, the amount of HCV antibodies in the sample is deduced by monitoring the competitive effect on the binding of a known amount of labeled antibody (or other competing ligand) in the complex.

Complexes formed comprising anti-HCV antibody (or, in the case of competitive assays, the amount of competing antibody) are detected by any of a number of known techniques, depending on the format. For example, unlabeled HCV antibodies in the complex may be detected using a conjugate of antixenogeneic Ig complexed with a label, (e.g., an enzyme label).

Immunassay for HCV Antigen(s)

In immunoassays, the test sample, typically a biological sample, is incubated with anti-HCV antibodies under conditions that allow the formation of antigen-antibody complexes. Various formats can be employed. For example, a “sandwich assay” may be employed, where antibody bound to a solid support is incubated with the test sample; washed; incubated with a second, labeled antibody to the HCV antigenic polypeptides, and the support is washed again (infra). HCV antigenic polypeptides are detected by determining if the second antibody is bound to the support. In a competitive format, which can be either heterogeneous or homogeneous, a test sample is usually incubated with antibody and a labeled, competing antigen is also incubated, either sequentially or simultaneously. These and other formats are well known in the art.

Kits suitable for immunodiagnosis and containing the appropriate labeled reagents are constructed by packaging the appropriate materials, including the polypeptides of the invention containing HCV epitopes or containing antibodies directed against HCV epitopes in suitable containers, along with the remaining reagents and materials required for performing the assay, as well as a suitable set of assay instructions.

Preparation of Anti-HCV Antibodies

According to the invention, HCV antigenic polypeptides, such as E2 and/or E1 glycoproteins, may be used as an immunogen to generate antibodies which recognize such an immunogen. Such antibodies include but are not limited to polyclonal, monoclonal, chimeric, single chain, Fab fragments, and an Fab expression library.

Various procedures known in the art may be used for the production of polyclonal antibodies to HCV antigenic polypeptides. For the production of antibody, various host animals can be immunized by injection with the HCV antigenic polypeptides, including but not limited to rabbits, mice, rats, tc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and otentially useful human adjuvants such as BCG (bacille Calmette-Guerin) and corynebacterium parvum.

For preparation of monoclonal antibodies directed toward HCV antigenic polypeptides any technique which provides for the production of antibody molecules by continuous cell lines in culture may be used. For example, the hybridoma technique originally developed by Kohler and Milstein (1975, Nature 256:495-497). According to the invention, human antibodies may be used and can be obtained by using human hybridomas (Cote et al., 1983, Proc. Nad. Acad. Sci. USA 80:2026-2030) or by transforming human B cells with EBV virus in vitro (Cole et al., 1985, in Monoclonal Antibodies and Cancer Therapy, Alan R. Liss, pp. 77-96).

Recombinant RV Vectors Mressing an HIV-1 Envelope Protein

In a preferred emodiment recombinant RVs expressing HIV-1 envelope protein is explained. To generate RV recombinant viruses expressing HIV-1 gp160, a new vector is constructed based on the previously described infectious RV cDNA clone pSAD-L16. (Schnell, et al., EMBO Journal, 13:4195-4203, 1994). Using site directed mutagenesis and a PCR strategy, the Ψ gene is deleted from the RV genome and a new transcription unit, containing a RV Stop/Start signal and two single sites (BsiWI and NheI), is introduced into the RV genome (see also Generation of recombinant vectors, supra). The resulting plasmid is designated pSBN (FIG. 1). The SBN RV-vector is recovered by the reported methods and displayed the same growth characteristics and similar viral titers as SAD-L16, indicating that neither the deletion of the Ψ gene nor the new transcription unit affected the RV vector (deleted). The HIV-1 envelope genes (NL4-3 and 89.6) to be expressed from SBN are generated by PCR and cloned between the BsiWI and NheI sites, resulting in the plasmids pSBN-NL4-3 and pSBN-89.6 (FIG. 1). All constructs are checked via DNA sequencing. It should be noted that foreign genes up to at least 4 kb are stable within the RV genome and a full length HIV-1 envelope protein is expressed from the recombinant RVs.

Recombinant RVs expressing either HIV-1_{NLA-3} or HIV-1_{89.6} envelope proteins are recovered by transfection of cells stably expressing the T7-RNA-polymerase with plasmids encoding the RV N, P, and L proteins along with a plasmid coding for the respective RV full-length anti-genomic RNA. Three days after transfection, supernatants of transfected cells are transferred to fresh cells and three days later analyzed by indirect immunofluorescence microscopy for expression of HIV-1 gp160. A positive signal for gp160 in cells infected with recombinant SBN-NL4-3 and SBN-89.6 confirmed the successful recovery of recombinant RVs expressing HIV-1 envelope protein. The recombinant RVs expressing HIV-1 gag are also constructed and recovered with the same procedure used for the recombinant RVs expressing HIV-1 envelope protein.

Growth Characteristics of Recombinant RVs

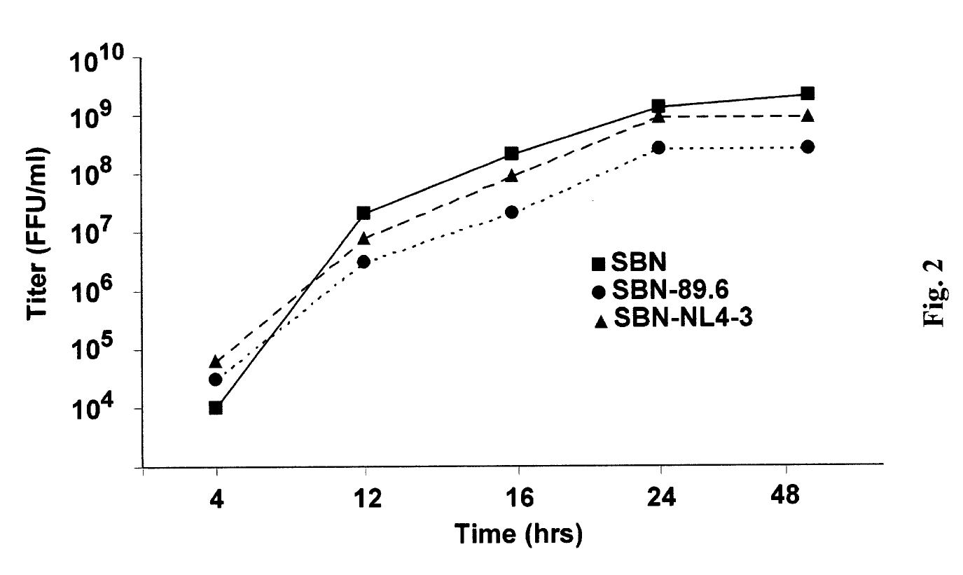

Growth characteristics of recombinant RVs expressing HIV-1 envelope protein are examined. A three-fold lower titer for SBN-NL4-3 and a 10-fold titer reduction for SBN-89.6 is noticed, as compared to wild-type SBN. To examine the differences in virus replication in detail, a one-step growth curve of the recombinant RVs is performed. BSR cells are infected with a MOI of ten to allow synchronous infection of all cells. After replacing the virus inoculum with fresh medium, viral titers are determined at the indicated time-points (FIG. 2). Both recombinant RVs expressing HIV-1 gp160 replicated at only a slightly reduced rate compared to wild-type RV, with the final titers being 2.3-(SBN-NL4-3) or 8-fold (SBN-89.6) reduced. The 20% longer genome size of the recombinant RVs cannot explain the slower growth of these viruses. A recombinant RV expressing a 1.9 kb gene (firefly luciferase) grew to wild-type RV titers. (Mebatsion, et al., Proceedings of the National Academy of Sciences of the United States of America, 93:7310-4, 1996).

Expression of Foreign Glycoprotein by Recombinant RVs

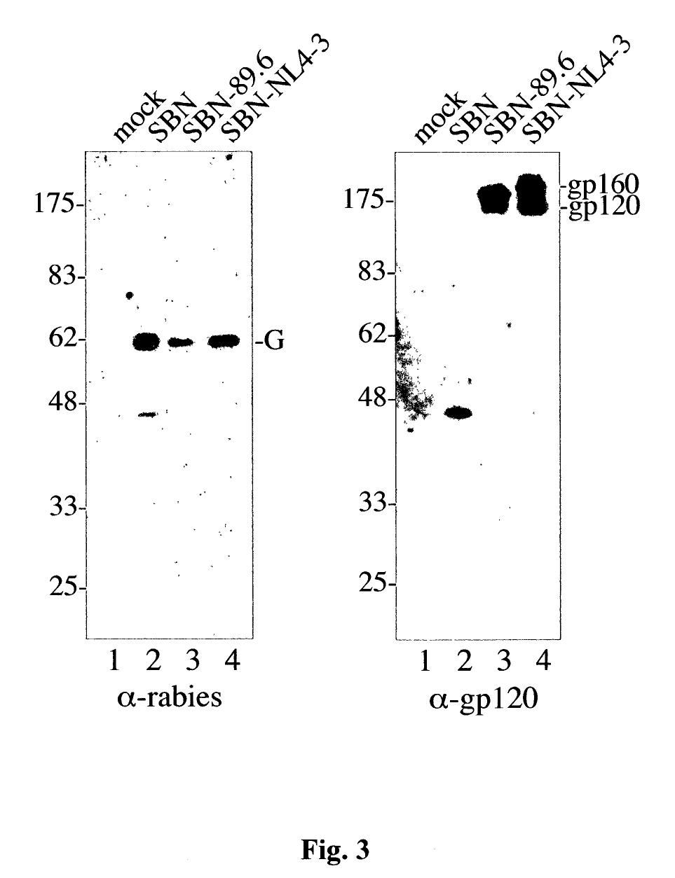

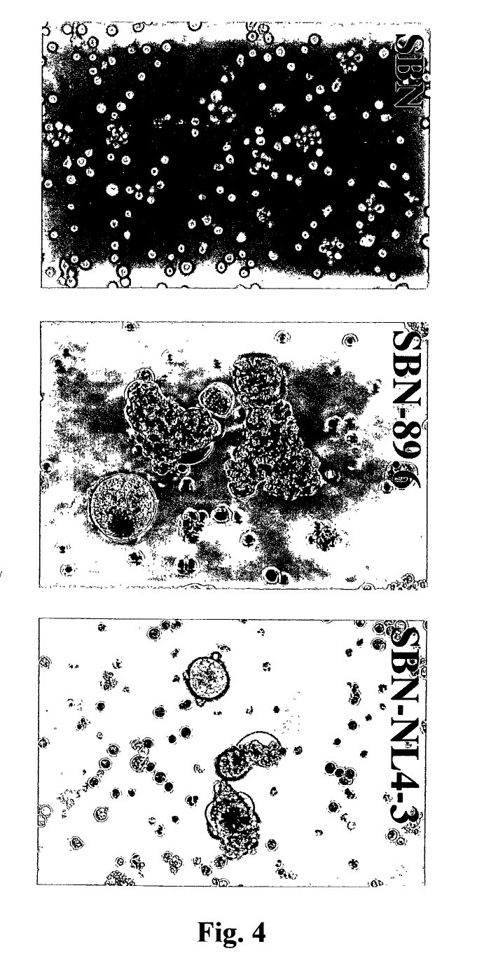

Expression of HIV-1 gp160 by recombinant RVs is also examined. To ensure the expression of HIV-1 gp160 by the recombinant viruses, cell lysates from recombinant RV infected cells are analyzed by Western immunoblotting with an antibody directed against RV (FIG. 3, a-rabies) or HIV-1 gp120 (FIG. 3, (X-gp120). Two bands of the expected size for HIV-1 gp160 and gp120 are detected in lysates from cells infected with SBN-89.6 or SBN-NL4-3 (FIG. 3, lanes 3 and 4), but are not observed in cell lysates of mock-infected or SBN infected cells (FIG. 3, lanes 1 and 2). The Western blot probed with an U.RV antibody confirmed that all viruses (FIG. 3, lanes 2, 3, and 4) infected the target cells.

Envelope Proteins Expressed in Recombinant RVs are Functional

To determine whether the expressed HIV-1 envelope protein is functionally expressed from RV, the recombinant RVs are analyzed in a fusion assay in a human T cell-line (Sup-Ti). This experiment confirmed that wild-type RV is able to infect and replicate in human T cell-lines. Because wild-type RV infects cells by receptor-mediated endocytosis, the RV glycoprotein (G) can only cause fusion of infected cells at a low pH. (Whitt, et al., Virology, 185:681-8, 1991). In contrast to wild-type RV, large syncytium-formation is detected in Sup-T1 cells 24 hours after infection with SBN-89.6 or SBN-NL4-3 (FIG. 4). These results indicate that the expressed HIV-1 envelope proteins are properly folded, transported to the cell surface, and are recognized by the HIV-1 receptor and coreceptor, CD4 and CXCR4.

Envelope protein from the dual-tropic HIV-1 strain (89.6) will induce cell fusion if coexpressed with CD4 and CCR5, whereas NL4-3 gp160 will only induce fusion on cells expressing CD4 and the HIV-1 coreceptor CXCR4. Infection of 3T3 murine cells expressing human CD4 does not result in cell fusion regardless of the recombinant RV used, whereas syncytium-formation is detected in 3T3 cells expressing CD4 and CXCR4 after infection with SBN-NL4-3 or SBN-89.6. As expected, only expression of HIV-1₈₉₆ envelope protein in 3T3 cells, expressing CD4 and CCR5, caused fusion of these cells.

Induction of a Humoral Immune Response in Mice

Anti-gp120 antibody response in mice infected with RV expressing HIV-1 gp160 is also analyzed. One likely requirement for a successful HIV-1 vaccine is the ability to induce a strong humoral response against the HIV-1 protein gp160. To determine whether the recombinant gp160 proteins expressed by recombinant RV are able to induce an anti-HIV-1 immune response, groups of five BALB/c mice are inoculated subcutaneously in both rear footpads with 10⁶ FFU of SBN, SBN-89.6, or 10⁵ FFU SBN-NL4-3. Mice are bled 11, 24, and 90 days after the initial infection with RV and the sera are analyzed by ELISA.

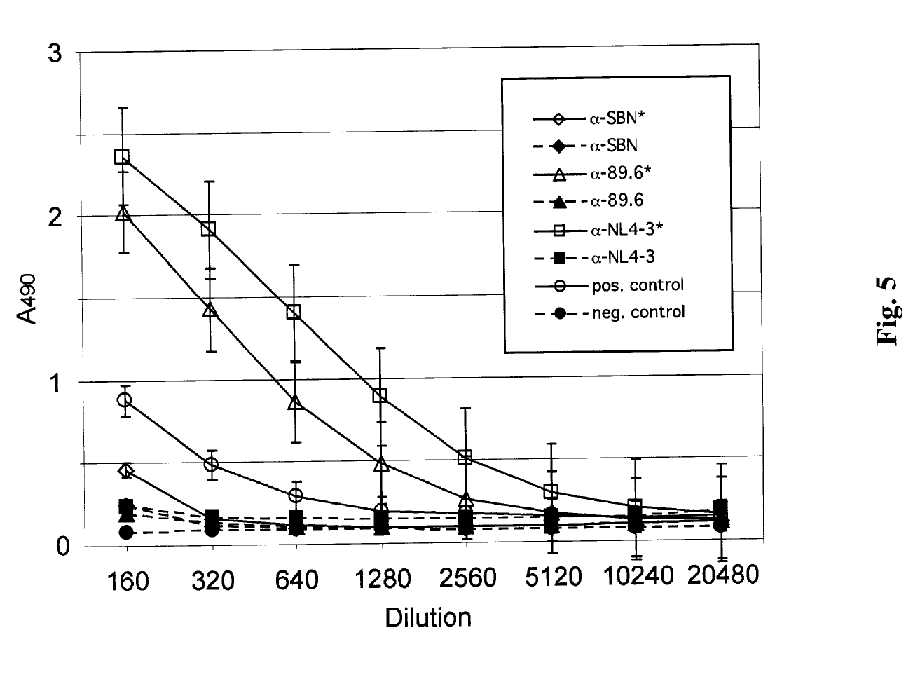

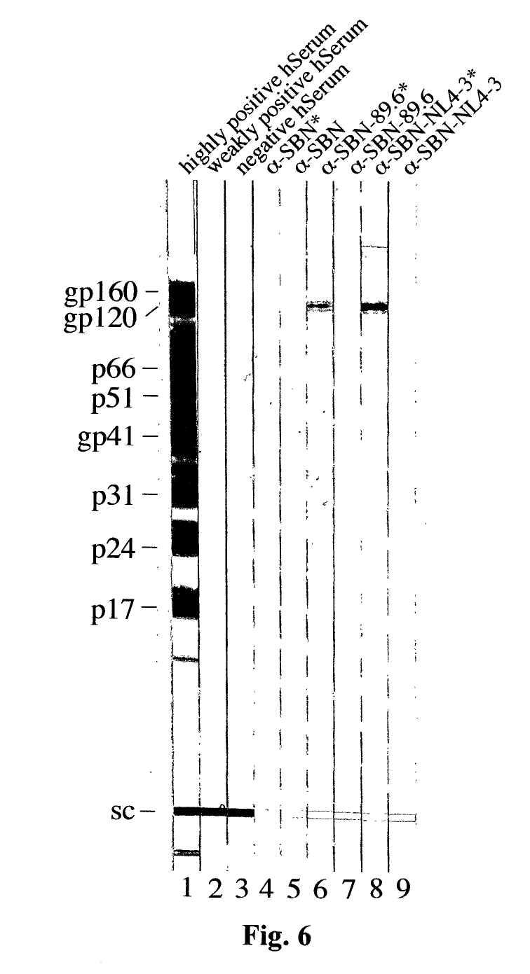

No response to the HIV-1 envelope is detected in the sera of immunized animals, but an ELISA using RV glycoprotein, instead of HIV-1 gp120, as an antigen confirmed the RV infection and detected high level of antibodies against RV as early as 11 days after infection. Several studies on viral vectors expressing HIV-1 gp160 indicated that a booster infection or a boost with a recombinant protein is necessary to induce detectable serum antibody response against HIV-1 envelope protein. The high antibody titer detected in the RV ELISA indicated that an additional infection with the recombinant RV would not be promising, therefore 3 out of 5 mice from every group were boosted with 10 μg of recombinant gp120 and gp41 in complete Freund adjuvant. Twelve days after the subunit boost, the mice are bled and the immune response is analyzed by an HIV-1 gp120 ELISA. The results demonstrate that an HIV-envelope subunit boost elicits a strong immune response against HIV-1 gp120 only in mice previously infected with SBN-89.6 or SBN-NL4-3 (FIG. 5). Wild-type RV (SBN) infected mice reacted only in the lowest serum dilution (1:160) after the boost. An ELISA specific for HIV-1 gp41 is negative for all mouse sera, even after the boost with recombinant HIV-1 gp120/gp41. These data are confirmed by Western blot analysis (FIG. 6). Only sera from mice infected with SBN-89.6 or SBN-NIA-3 and subsequently boosted with recombinant protein are able to react with gp120, whereas all other sera failed to detect any HIV-1 protein. None of the sera had gp41-specific bands, even with a gp41 subunit immunization.

Induction of Neutralizing Antibodies

An experiment is also carried out to see whether primary virus infection followed by recombinant protein boost induces neutralizing antibodies against HIV-1. In this experiment, HIV-1 neutralizing antibody (NA) titers are determined in MT-2 cells by a vital dye staining assay using HIV-1_{NL4-3}. The mouse serum is able to neutralize a tissue culture laboratory adapted (TCLA) HIV-1_{NL4-3} strain at a 1:800 serum dilution after immunization with SBN-NL4-3 and an envelope subunit booster injection of recombinant gp120 (IIIB strain), whereas immunization with SBN-N4-3 did not induce detectable neutralizing antibody. These results are confirmed in two independent experiments. The sera from wild-type RV (SBN) infected mice which received a recombinant gp120 boost displayed only a very low NA titer of 1:50 (Table 1). These results indicate that a boost injection with recombinant gp120 following the priming with recombinant RV expressing HIV-1 gp160 elicits high titers of NA.

TABLE 1
Neutralizing antibody totres of sera from mice infected with different
RVs followed by boost injection of recombinant HIV-1 gp 120/gp 41.

| Immunization with *boost | | |
|---|---|---|
| injection of recombinant HIV-1 | HIV-1_{NL4-3} Neutralizing Antibody Titer | |
| gp 120/gp 41 | Experiment I | Experiment II |
| SBN-NL4-3 | <1:50 | 1:50 |
| SBN-NL4-3* | 1:800 | 1:800 |
| SBN* | <1:50 | <1:50 |

The results presented herein demonstrate that a recombinant RV expressing a full-length HIV-1 envelope protein is generated. The foreign gene is stably expressed by replication competent RV and induces a strong humoral response in mice against HIV-1 envelope protein after infection with recombinant RV and a single subsequent boost of HIV-1 gp120 protein. Infection of mice with recombinant RV expressing HIV-1 gp160 results in a strong priming of the immune system, as indicated by vigorous humoral responses after a single boost with HIV-1 gp120 protein or gp41. Thus, boosting with another recombinant RV using a different viral glycoprotein for infection of the mice, or recombinant VSV expressing HIV-1 gp160 can be tested for an even stronger response.

Induction of Long-Lasting HIV-1 gp160-speciflc CTL.

Recombinant RV expressing HIV-1 envelope protein from a laboratory-adapted HIV-1 strain (NL4-3) and a primary HIV-1 isolate (89.6) show that RV-based vectors are excellent for B cell priming (supra). (Schnell, M. J., et al., Proc. Natl. Acad. Sci. USA, 97:3544-3549, 2000.). The present invention further relates to the memory CTL response against HIV-1 envelope protein expressed by the attenuated RV-based vectors. As noted, increasing evidence suggests that the induction of a vigorous, long-lasting CTL response is an important feature for a successful HIV-1 vaccine.

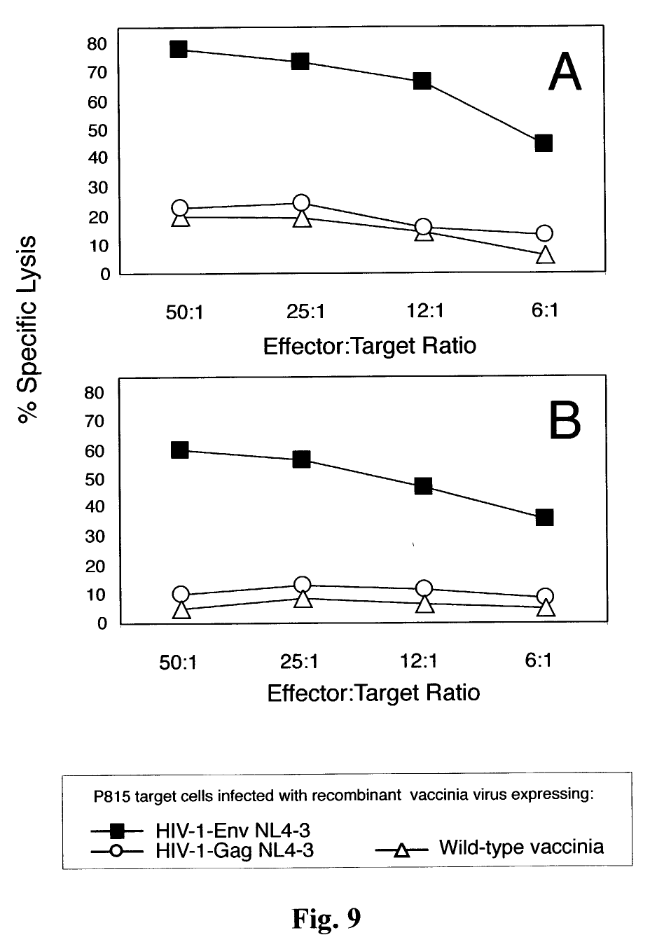

To analyze the potency of RV-based vectors to induce a cytotoxic response against HIV-1, six mice were immunized with 2×10⁷ foci forming units (FFU) of the recombinant RV expressing HIV-1_{NL4-3} envelope protein (SBN-NL4-3) (supra and infra). Three mice are sacrificed 105 or 135 days after infection and the spleens are removed. One third of the splenocyte cultures are infected with a multiplicity of infection (moi) of 1 with a recombinant vaccinia virus expressing HIV-1_{NL4-3} gp160 for 16 hours, deactivated using Psoralen and UV treatment, and added back to the culture as presenter cells. Stimulated effector cells are analyzed 7 days after activation for their ability to kill P815 target cells infected with vaccinia wild-type virus, a recombinant vaccinia virus expressing HIV-1_{NL4-3} gp160 or HIV-1 Gag. As can be observed in FIG. 9, a strong cytotoxic response is detected only against P815 target cells infected with the recombinant vaccinia virus expressing HIV-l envelope protein. Only a low percentage of lysis is observed for P815 cells infected with the other two vaccinia viruses. Of note, these responses are achieved after a single inoculation with recombinant RV expressing HIV-1 envelope protein, which indicates that RV-based vectors are able to induce long-lasting CTLs after a single vaccination.

CTLs from HIV-1 gp160 Immunized Mice Cross-Kill Target Cells Expressing a Heterologous HIV-1 Envelope Protein

There is a significant difference in HIV-1 envelope amino acid sequences but cross-protection between divergent viruses will be a likely requirement for a protective HIV-1 vaccine. To analyze the potency of the vaccine candidate to induce cross-reactive CTLs against gp160 from different HIV-1 strains, splenocytes from mice immunized with a recombinant RV expressing HIV-1 gp160 are screened against P815 target cells expressing homologous and heterologous HIV-1 envelope proteins. For this approach, two groups of six mice are immunized intraperitoneally (i.p) either with 2×10⁷ recombinant RV expressing HIV-1 gp160 from a laboratory-adapted, CXCR4-tropic (NL4-3) or a dual-tropic (CXCR4 and CCR5) isolate (89.6).

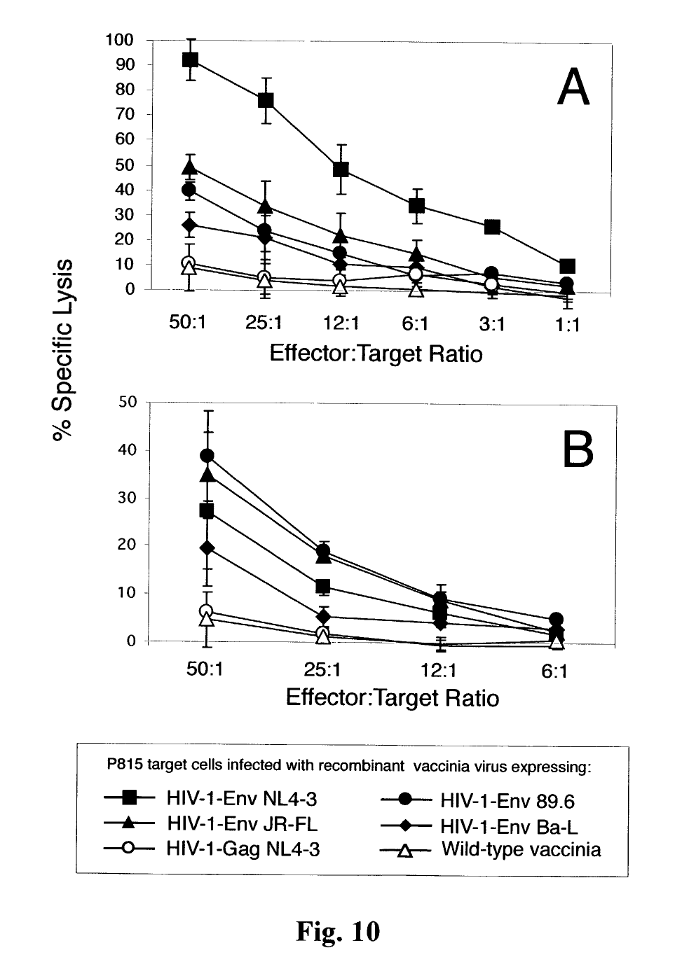

Three and five weeks after the immunization, three mice from each group are sacrificed, the spleens are removed, and the pooled splenocytes are stimulated with a recombinant vaccinia virus expressing the homologous HIV-1 envelope protein (NL4-3 or 89.6). Seven days after the stimulation, effector cells are analyzed for their ability to lyse P815 cells infected with recombinant vaccinia viruses expressing HIV-1 envelope protein from the laboratory-adapted, CXCR4-tropic HIV-1 strain (NL4-3), the dual-tropic strain (89.6), and two primary, CCR5-tropic HIV-1 strains (Ba-L and JR-FL). The results from two different, independent experiments are shown in FIG. 10A for mice immunized with a RV expressing HIV-1_{NL4-3} Env and in FIG. 10B for mice immunized with RV expressing HIV-1_{89.6} Env. As expected, a strong, specific lysis of P815 cells expressing the homologous antigen is observed for both groups. More striking, these effector cells are able to cross-kill P815 target cells expressing heterologous HIV-1 envelope proteins. Activated splenocytes from SBN-NL4-3 immunized mice achieved a specific lysis of P815 cells expressing gp160 JR-FL or 89.6 in the 40% range at an effectortarget (E:T) ratio of 50:1 and are also able to cross-kill target cells expressing HIV-1_{Ba-L} gp160. Cross-killing is also observed with effector cells from SBN-89.6 primed mice. P815 target cells are lysed in the same range as observed for activated splenocytes from mice immunized with SBN-NL4-3, but lysed only about 20% P815 cells expressing HIV-1_{NL4-3}. These data indicate that CTLs against HIV-1 gp160 induced by RV-based vectors may be directed against different epitopes within the HIV-1 envelope protein.

HIV-1-specific CTL Activity is Mediated by CD8⁺ T-cells

The phenotype of the T-cell subpopulation mediating cytolytic activity is assessed by selective depletion. Three mice are immunized with 2×1 FFU of recombinant RV expressing HIV-I_{NL4-3} envelope protein, eighteen weeks later the spleens are removed. Splenocytes are re-stimulated with a recombinant vaccinia virus expressing the homologous HIV-1 envelope protein for 7 days. Immuno-magnetic bead cell separation is completed to both deplete and positively isolate CD8⁺ T-cells from the activated splenocyte culture. Chromium release assays are completed using cultures depleted of CD8⁺ T-cells (CD8⁻), cultures of isolated CD8 cells (CD8⁺) or unprocessed cultures (CD8⁺/CD8⁻).

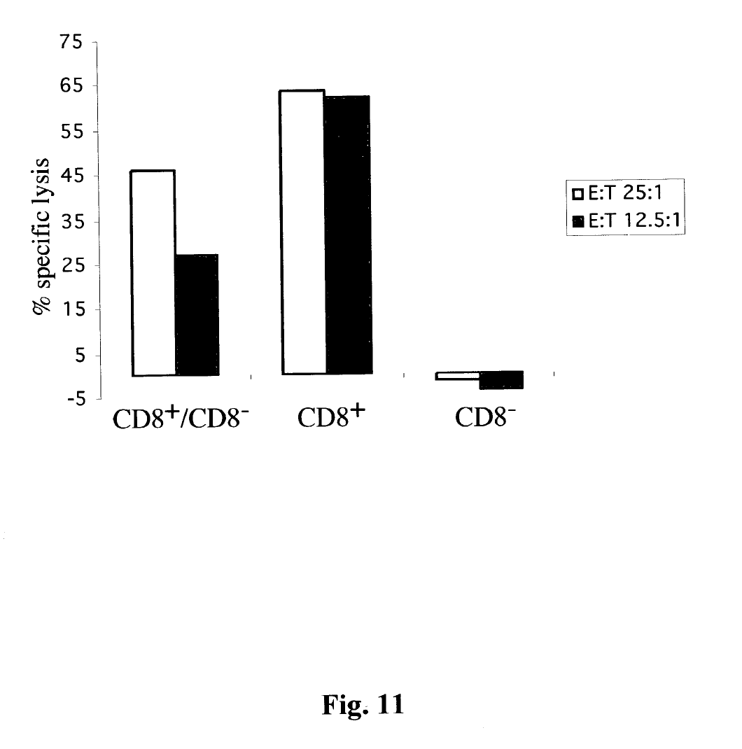

P815 target cells are infected with vaccinia virus expressing HIV-1_{NL4-3} gp160 or HIV-1 gag. As illustrated in FIG. 11, the CD8⁺ T-cell depleted cultures show no activity while the CD8⁺ T-cell enriched and unprocessed cultures show high specific lysis at E:T ratios of 25:1 and 12.5:1, respectively. Indeed, the CD8⁺ T-cell enriched population is also enriched in lytic units, as the CTL activity is still on a plateau at 12.5:1, in contrast to the unselected population. These data indicate that the cytolytic activity is mediated by the CD8⁺ T-cell sub-population. Furthermore, these results imply that in addition to antibodies, recombinant RV vectors also generate long-lived anti-HIV-1 CD8⁺ T-cell responses.

Construction of Recombinant RVs Expressing HCV Structural Proteins.

E1 and E2 are present on the surface of HCV virions (Dubuisson, 2000). Furthermore, HCV E2 has been reported to interact with CD81, a potential receptor for HCV (Pileri et al., 1998). The present invention provides a Rhabdovirus-based vector that expresses E1 and/or E2 for use as an HCV vaccine wherein HCV glycoprotein(s) are presented to the immune system for the generation of both a cellular and an immune response.

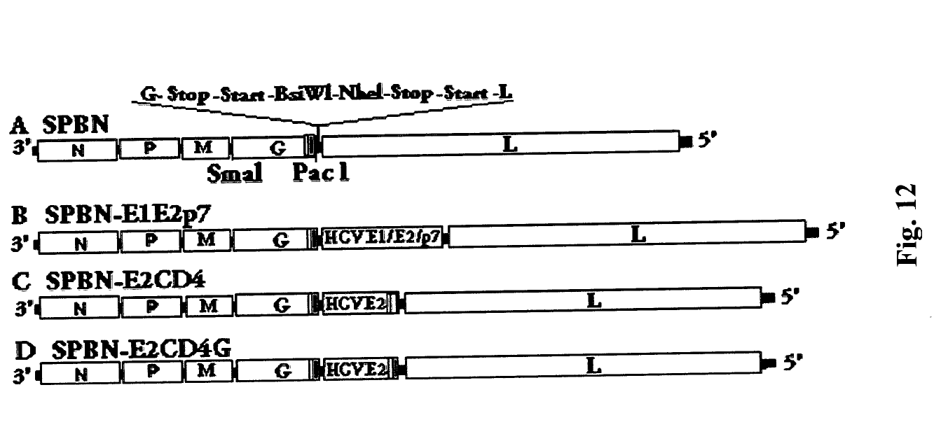

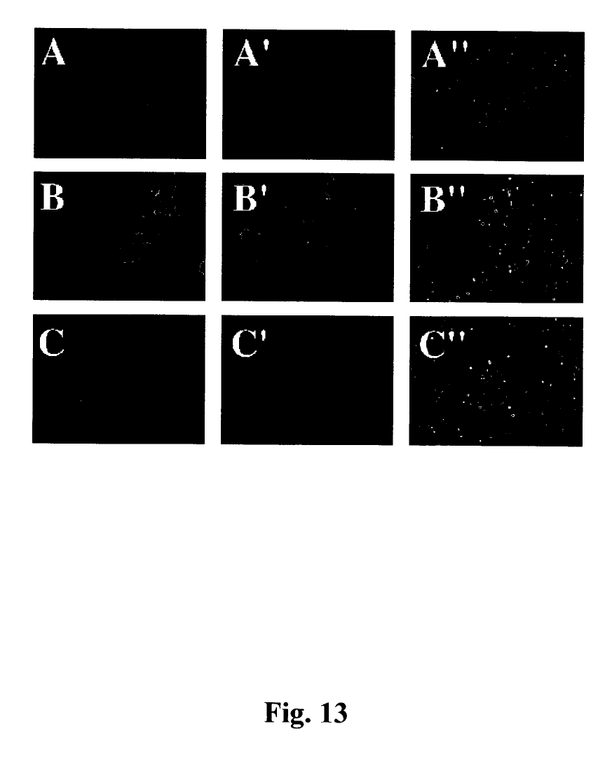

To generate RV recombinant viruses a RV vaccine strain-based vector is used with a new RV transcription unit, containing a RV Stop/Start signal and two single sites (FIG. 12 and supra). The gene encoding the HCV E1, E2, and p7 proteins, to be expressed from SPBN, were generated by PCR and cloned between the BsiWI and NheI sites, resulting in plasmid pSPBN-EIE2p7.

To analyze if the expression of the HCV E2 on the surface of the infected cell enhances HCV immunogenicity, two similar RV recombinant viruses are generated. One contains the gene encoding the ectodomain of HCV E2, with an 85 amino acid deletion at the carboxy-terminal end, fused to the transmembrane domain (TMD) and cytoplasmic domain (CD) of human CD4 (CD4). The second recombinant virus contains the gene encoding the ectodomain of HCV E2, with an 85 amino acid deletion at the carboxy-terminal end, fused to the TMD of CD4 and the CD of RV G. These constructs were PCR amplified and the resulting products cloned into the BsiWI/NheI sites of pSPBN, resulting in pSPBN-E2CD4 and pSPBN-E2CD4G, respectively (FIG. 12). The pSPBN-E2CD4G was constructed on the basis of a previous finding that the RV CD is required for incorporation of a foreign glycoprotein into RV virions (Mebatsion and Conzelmann, 1996c; Mebatsion et al., 30 1997).

As shown previously, RV vectors stably express large foreign genes (McGettigan et al., 2001a; Mebatsion et al., 1996; Schnell et al., 2000). The infectious RVs were detected in tissue culture supernatants of cells transfected by standard RV recovery protocols for pSPBN, pSPBN-E1E2p7, pSPBN-E2CD4, and pSPBN-E2CD4G (Finke and Conzelmann, 1999). In contrast to the previously constructed recombinant RVs expressing HIV-1 gp160 (Schnell et al., 2000; supra), the recombinant RVs expressing HCV proteins grew to the same (or greater) titers as SPBN, which were at least 10⁸ FFU.

Expression of HCV Glycoproteins by Recombinant RVs.

The HCV envelope proteins E1 and E2 interact to form a non-covalent heterodimeric complex, which is retained in the endoplasmic reticulum (ER). The 10 chimeric HCV E2 protein containing the transmembrane domain (TMD) and cytoplasmic domain (CD) of CD4 is transported to the cell surface (Dubuisson, 2000). To ensure that the replacement of the CD of E2CD4 with that of RV G did not interfere with the surface expression of the recombinant protein, BSR cells were infected with SPBN-E1E2p7 (FIG. 13, Panels A, A′, A″), SPBN-E2CD4G (FIG. 13, Panels B, B′, B″) or SPBN (FIG. 13, Panels C, C′C″) at a multiplicity of infection (MOI) of 0.1. Cells were fixed 48 hours later with paraformaldehyde. Infected cells were analyzed directly by immunofluorescence microscopy with a monoclonal antibody directed against HCV E2 (FIG. 13 panels A′, B′, C′). Alternatively, cells were permeabilized with Triton X-100 for internal staining with an antibody against HCV E2 (FIG. 13 panels A, B, C) or RV N protein (FIG. 13 panels A″, B″, C″). The results indicate that the chimeric E2 protein containing the CD4 TMD and the RV G CD is transported to the surface of the infected cell. Furthermore, immunostaining with a conformation-sensitive monoclonal antibody revealed that the recombinant HCV E2 protein is properly folded.

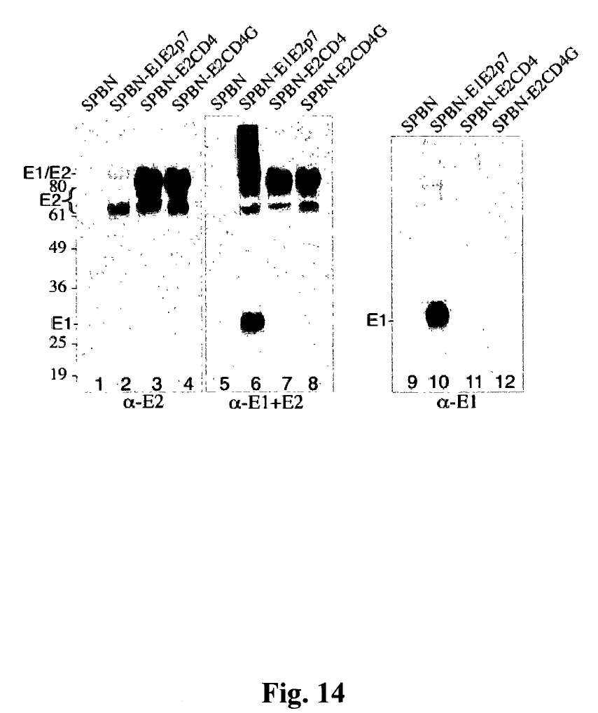

To analyze the expression and processing of HCV E1 and E2 by the recombinant viruses, lysates from cells infected with recombinant RVs were separated by SDS-PAGE under reducing conditions, followed by Western immunoblotting using HCV E2 specific monoclonal antibodies (H-52). Cell lysates infected with SPBN-E1E2p7 had two bands of the expected size for the uncleaved precursor of the E1E2 polyprotein and for the cleaved E2 (FIG. 14, lane 2). A band similar in size to the E2 expressed by SPBN-E1E2p7 was also detected in lysates from SPBN-E2CD4 and SPBN-E2CD4G infected cells. In addition, a more diffuse, slow-migrating band, which was not observed for wild-type E2, was detected for both chimeric E2 proteins (E2CD4, containing the CD4 TMD and CD; and E2CD4G, containing the CD4 TMD and the RV G CD).

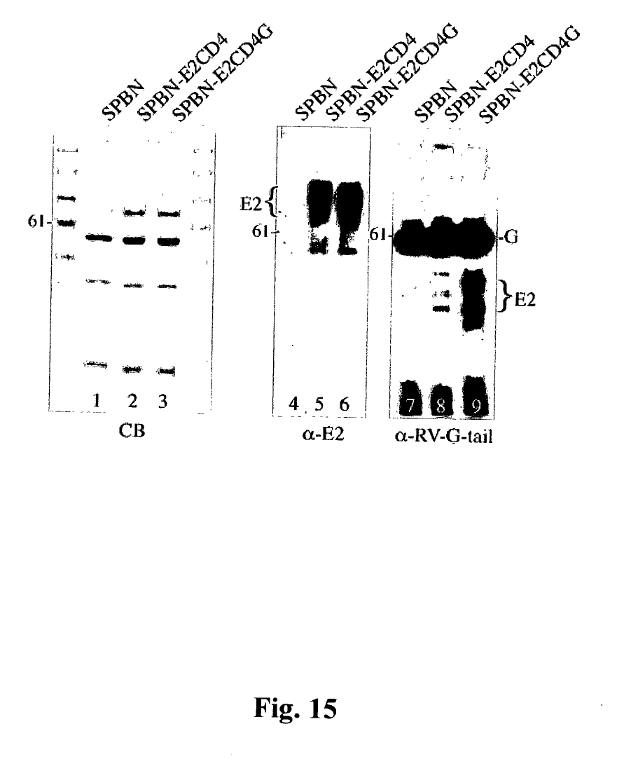

Previous experiments by Dubuisson and coworkers suggested that the slower-migrating band of E2 corresponded to E2 molecules no longer retained in the endoplasmic reticulum (ER), but were processed by Golgi enzymes resulting in removal of their glycans (Cocquerel et al., 2000). It is interesting to note that only the slower migrating form of E2 is incorporated into RV virions (FIG. 15, lanes 5 and 6), thereby supporting the theory that these molecules reach the cell surface. In contrast, the faster migrating band is retained in the ER.

Immunoblotting with the monoclonal antibody A4, directed against HCV E1 (Dubuisson et al., 1994), detected a band of about 27 kD, as expected for HCV E1, only in cell lysates infected with the recombinant RV SPBN-E1E2p7 (FIG. 14, lanes 6 and 10). This result confirmed the cleavage of the E1E2p7 precursor protein even in a non-human cell-line (BSR). Infection with all four recombinant RVs was confirmed with a human polyclonal serum directed against RV G protein.

Recombinant HCV E2 is Incorporated into RV Virions.

A recombinant virion containing HCV E2 is provided by the present invention and is useful for producing E2 antigen for diagnostic use, as well as for a killed vaccine against HCV. To analyze incorporation of the chimeric E2 proteins into RV particles, BSR cells were infected with SPBN, SPBN-E2CD4 and SPBN-E2CD4G with a MOI of 0.1. Three days after infection, virus was purified from the supernatants of infected cells by a 20% to 70% density sucrose gradient. Viral proteins were separated by SDS-PAGE and detected by Coomassie blue staining.

The results in (FIG. 15, lanes 1-3) showed equal amounts and the same pattern of the RV proteins for all three recombinant viruses, but no additional protein of the expected size for the HCV E2 could be detected in the viral particles. The lack of detection of E2 may be due to E2 incorporation at low levels or that E2 is migrating through the gel as a more diffuse band then the other RV proteins due to the presence of multiple O- and N-linked glycans.

The recombinant virions were then analyzed by Western blotting using an antibody directed against E2. The recombinant E2 was readily detected in both SPBN-E2CD4 and SPBN-E2CD4G particles (FIG. 15, lanes 5 and 6), whereas no signal was detected for SPBN (FIG. 15, lane 4) or SPBN-E1E2p7. It was surprising that both E2CD4 and E2CD4G were incorporated into RV particles since an earlier finding by Mebatsion et al. indicated that the RV G CD is a requirement for incorporation of a foreign glycoprotein into RV virions (Mebatsion et al., 1996). This is not the case for HCV E2, as quantification of E2 indicated that the content of the recombinant E2CD4 was at least 60% of E2CD4G.

The presence of the RV G CD in the HCV envelope protein expressed by SPBN-E2CD4G was also verified by Western blotting using an antibody specific for the RV G CD. Previous studies with this antibody showed that recombinant E2CD4G co-migrates with RV G, which made it impossible to distinguish between the two proteins. RV G contains only three to four N-linked glycosylation sites, whereas HCV E2 is a heavily O- and N-glycosylated. Therefore, the RV virions were digested with N-glycosidase F to remove the N-glycan chains. As illustrated in FIG. 15, lane 7-9, the anti-RV G antibody detected a band of similar size and intensity of deglycosylated RV G, whereas two prominent additional bands were detected in virions containing E2 envelope protein with the RV G.

Reactivity of Recombinant RV Virions with Human Sera.

Recombinant HCV E2 is primarily produced by transfection of cells with plasmids encoding a naive E2 or a truncated form of HCV E2, which is secreted in the tissue culture supernatant. In both cases, only small amounts of protein are produced. On the other hand, recombinant RVs are easy to grow and purify and 1 mg RV G protein can be extracted from 1 liter of tissue supernatants of RV infected cells. In addition, RV virions are readily deactivated prior to purification and, therefore, handling infectious material is limited to the growth of the viruses.

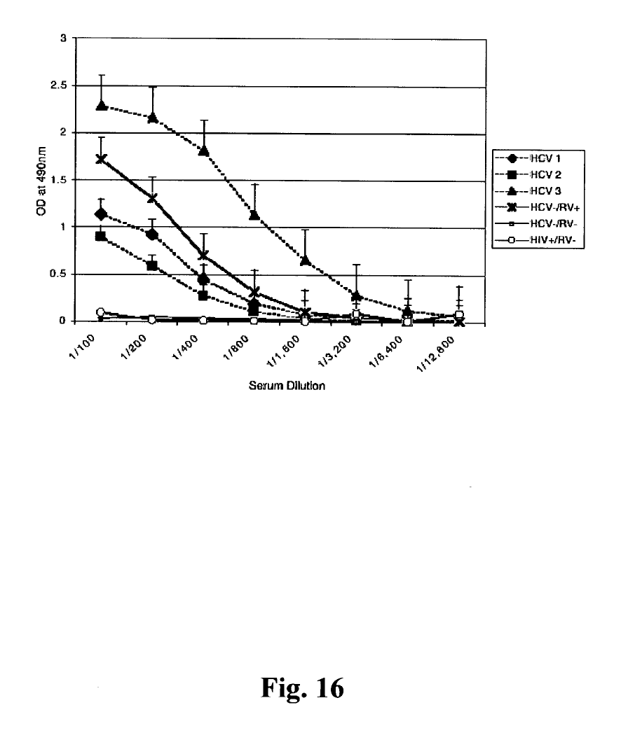

To analyze the antigenicity of the recombinant RV particles containing HCV E2, ELISA plates were coated with recombinant HCV E2 glycoprotein derived from purified SPBN-E2CD4G virions. The results (FIG. 16) indicate that the sera from three randomly chosen HCV-positive patients had high E2-specific ELISA titers, between 1:400 to 1:1,600 (FIG. 16, HCV1-3), whereas pooled human control sera from HCV- and RV-negative donors, and a serum from a HIV-1-positive patient, did not react (FIG. 16).

A control serum from a RV-vaccinated person showed a similar ELISA titer to that of the HCV-positive patients due to the presence of the RV G in the recombinant SPBN-E2CD4G virion used to coat the plates. (FIG. 16, HCV−/RV+). Only the sera from the RV vaccinated donor reacted with the control ELISA plates, coated with SPBN derived glycoprotein. As expected, the sera from the HCV patients did not react with the SPBN coated plates. Thus, the present invention provides recombinant RVs as a quick and easy tool to screen for seroconversion against E2 in HCV-infected individuals.

Recombinant RVs Expressing HCV Glycoproteins are Immunogenic in Mice. Induction of a Humoral Immune Response

The immune responses which may protect humans from HCV infection are not well-defined, but it is likely that both cellular and humoral responses will be required for protection of infection or clearence of HCV. To analyze the immunogenicity of the RV vector expressing HCV proteins, a group of ten female BALB/c mice were infected with 1×10⁷ FFU of SPBN-E2CD4G, a group of five mice with an equal amount of the RV vector SPBN, and left five mice uninfected.

Fourteen days post immunization, all mice were bled and sera analyzed by ELISA using recombinant HCV E2. No E2-specific antibodies were detected. Previous experiments with recombinant RVs expressing HIV-1 gp160 indicated that the induction, of a humoral response against HIV-1 gp160 required a boost with recombinant HIV-1 gp120 (Schnell et al., 2000; supra). Therefore, the mice were given a boost vaccination using killed RV particles derived from SPBN-E2CD4G infected cells were used as a source of recombinant HCV E2. A group of five mice primed with live SPBN-E2CD4G and boosted with killed SPBN particles served as a control.

Ten days later, mice were bled and sera analyzed by an HCV-specific ELISA. E2-specific antibodies were expected in the sera of mice primed with live SPBN-E2CD4G and boosted with killed SPBN-E2CD4G, but only two out of five mice had E2-specific antibodies. Of note, no adjuvant was used for the immunization with the killed virions, which may explain why only a portion of the mice developed antibodies directed against HCV E2.

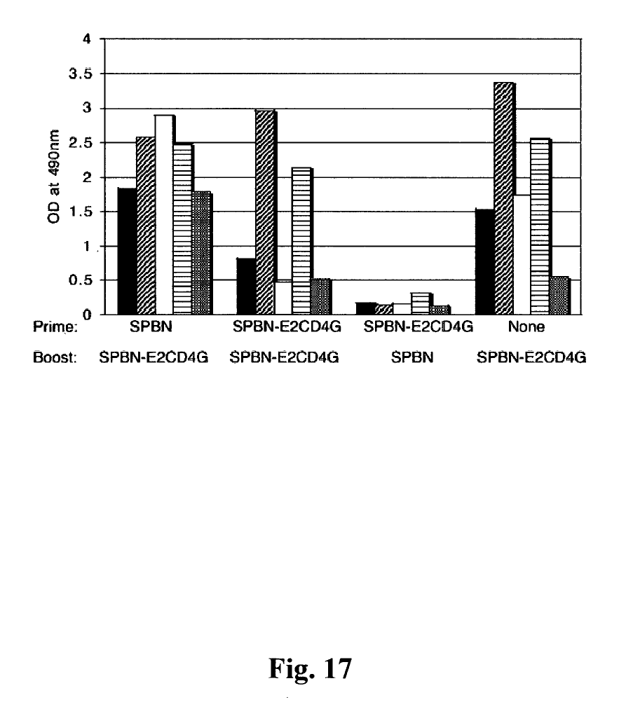

To analyze if a second inoculation with the same killed RV virions would induce a higher rate of serocoversion against HCV E2, mice from each group received a second immunization with the same killed virions that were used for the first immunization. Ten days later, the mice were bled and E2-specific ELISAs performed. The results (FIG. 17) show that all mice boosted with the killed virions containing the HCV E2 seroconverted, whereas sera from SPBN-E2CD4G primed mice that were boosted twice with killed SPBN virions were negative. These results indicate that two inoculations with inactivated RV virions containing chimeric HCV E2 are able to induce a potent humoral response directed against HCV E2. Of note, priming with the recombinant RV SPBN-E2CD4G did not result in a stronger B cell response against HCV E2, as seen in unprimed or SPBN primed mice.

Induction of a Cellular Immune Response

In contrast to HIV-1 gp160 (supra), limited information is available for specific CTL epitopes of HCV glycoproteins in mice. To analyze if a single inoculation with the RV-based vaccine vehicle expressing the HCV glycoproteins E1 and E2 is able to induce a cellular response against HCV E2, ten female BALB/c mice were vaccinated with 10⁷ FFU of SPBN-E1E2p7 and spleens were harvested 11 weeks later. Splenocytes were cultured and stimulated for 7 days with an E2-specific peptide (1323), and T-Stim was added as a source of IL-2.

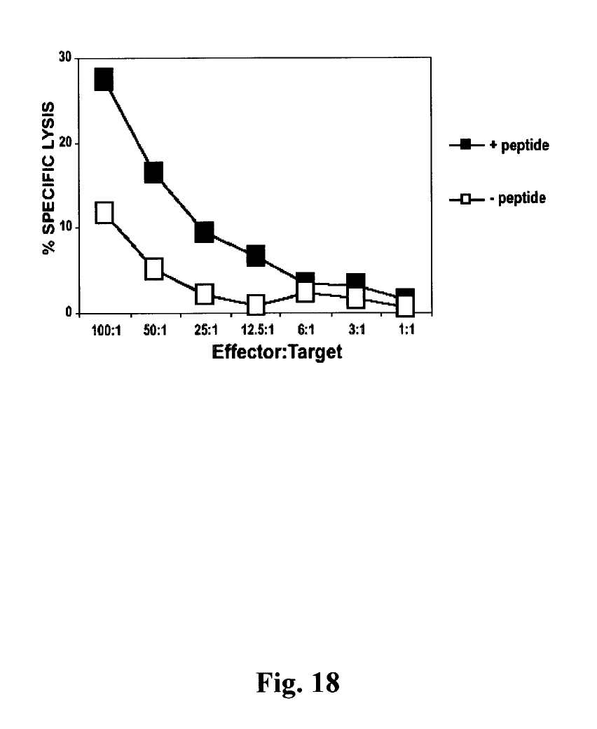

On the day of the assay, target cells were pulsed both with and without an E2 peptide (1323) and labeled with Cr⁵¹. Effectors and targets were incubated together at several ratios for four hours. Specific lysis was detected in a broad range of a effectortarget ratios of 100:1 to 12.5:1 (FIG. 18), indicating that a single inoculation with the recombinant RV expressing HCV-E2 of the present invention induces a long-lasting, antigen-specific cellular immune response.

Construction of Replication-Competent RV Expressing HIV-1 Gag Protein.

Both long-lasting and vigorous humoral and cellular immune responses were observed against the HIV-1 envelope proteins in vaccinated mice (supra). Another important target for a vaccine against HIV-1 is HIV-1 Gag, one of the most conserved proteins of HIV-1. In contrast to the surface envelope protein gp120, the Gag protein is a matrix protein and this protein makes up the viral nucleoocapsid.

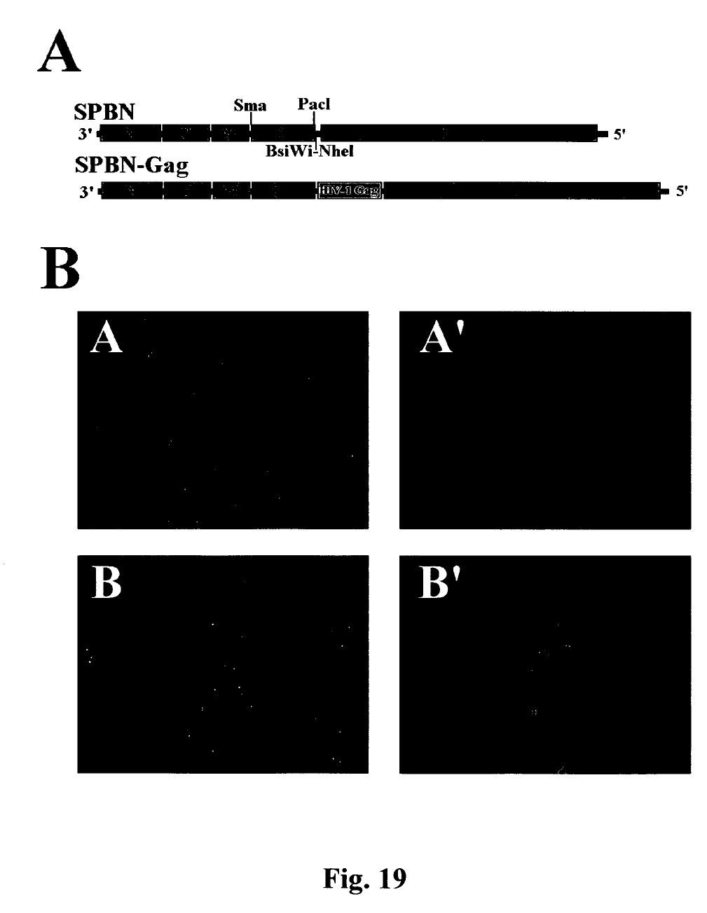

The construction of a recombinant RV vaccine-based vector SBN that contains a synthetic RV transcription unit and two single restriction sites downstream of the glycoprotein (G) gene has been described (supra). Using site-directed mutagenesis and a PCR strategy, a new single Pacl restriction site downstream of the coding region of the RV glycoprotein (G) gene was introduced into the pSBN cDNA resulting in pSPBN (FIG. 19A). To construct a recombinant RV expressing HIV-1 Gag, the HIV-1 NL4-3 gag coding region was amplified by PCR and cloned into pSPBN using the BsiWI and NheI sites. The resulting plasmid was designated pSPBN-Gag (FIG. 19A). Recombinant replication-competent RVs SPBN and SPBN-Gag were recovered by transfection of BSR cells stably expressing the T7-RNA-polymerase with plasmids encoding the RV N, P, and L proteins along with a plasmid coding for the respective RV full-length anti-genomic RNA (supra). Three days after transfection, supernatants of transfected cells were transferred to fresh cells and three days later analyzed by indirect immunofluorescence microscopy for the presence of infectious RVs. A positive signal for RV nucleoprotein confirmed the successful recovery of both recombinant RVs SPBN and SPBN-Gag. In contrast to the previously recovered recombinant RVs expressing HIV-1 envelope protein (supra), SPBN and SPBN-Gag grew to similar titers as the parental vector SBN which were at least 2×10⁸.

Expression of HIV-1 Gag Protein in Infected Cells.

To ensure expression of HIV-1 Gag from the recombinant RV, HeLa cells were infected with SPBN or SPBN-Gag with a MOI of 0.1 and the infected cells were analyzed by immunofluorescence 48 hours after infection. As shown in FIG. 19B, expression of RV nucleoprotein (N) was detected in cells infected with SPBN and SPBN-Gag (FIG. 19, panels A and B), whereas expression of HIV-1 Gag could only be detected in SPBN-Gag infected cells (FIG. 19, panel B′).

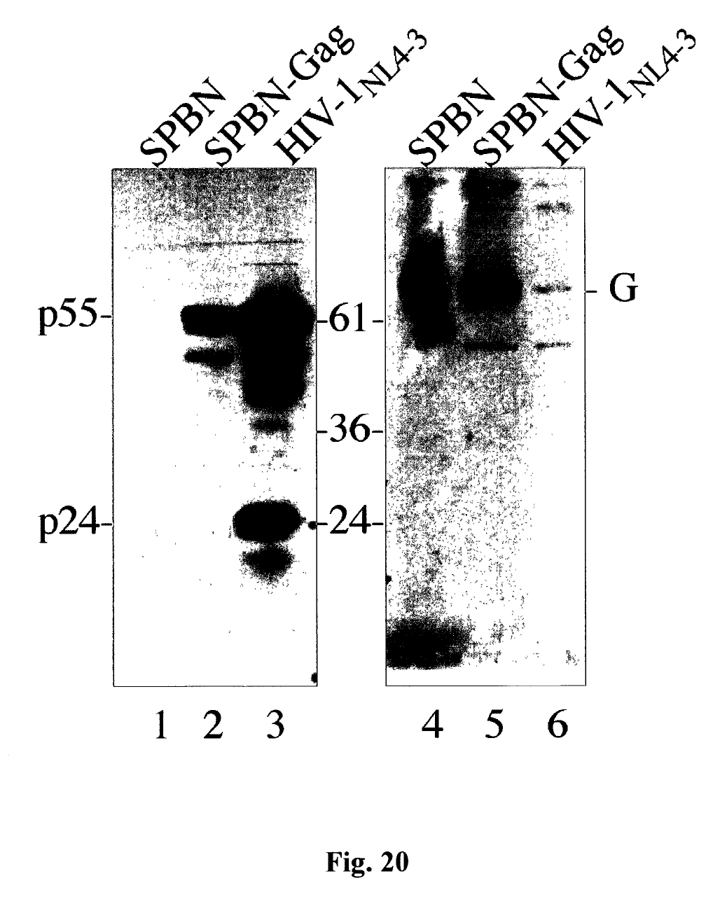

In the next step cell lysates from HeLa cells infected with SPBN or SPBN-Gag were analyzed by SDS-PAGE followed by immunoblotting with a human monoclonal directed against HIV-1 capsid protein p24 (FIG. 20 lanes 1-3) or a polyclonal rabbit antibody against RV G (FIG. 20 lanes 4-6). A strong signal identified a protein at the expected size for the HIV-1 Gag precursor p55 in the case of SPBN-Gag infected cell (FIG. 20, lane 2), whereas no HIV-1 Gag protein was detected in cells lysates of SPBN infected cells (FIG. 20, lane 1). In cell lysates from control cells infected with HIV-1, multiple protein bands were detected which represent proteolytic cleavage products of the HIV-1 p55 precursor protein (FIG. 20, lane 3). Because SPBN-Gag does not contain the HIV-1 protease, the smaller protein bands detected in SPBN-Gag infected cells are those derived from early termination and/or internal initiation of p55 translation.

Infection of Human Cells with Recombinant RV Expressing HIV-1 Gag Results in the Release of Immature HIV-1 VLPs.

To quantify expression of HIV-1 Gag by the recombinant RV SPBN-Gag, cells were infected with SPBN or SPBN-Gag at a MOI of 1 and cell culture supernatants and cell lysates were analyzed 48 hours later by a p24 antigen capture ELISA. The results shown in Table 2 indicate the efficient production and release of HIV-1 p55 in the range of 2 to 4.5 ng/ml for both BSR and HeLa cells infected with SPBN-Gag. No p24 antigen was detected on control cells infected with the RV expression vector SPBN (Table 2).

TABLE 2
Quantification of HIV-1 Gag in lysates and supernatants from SPBN or
SPBN-Gag infected cells. BSR or HeLa cells were infected with SPBN or
SPBN-Gag for 48 hours and p24 antigen was quantified using a p24
antigen ELISA assay.

| Cell line | SPBN-Gag | SPBN |
|---|---|---|
| HeLa, lysates | 2.1 ng/ml | <0.1 ng/ml |
| HeLa, supernatants | 4.5 ng/ml | <0.1 ng/ml |
| BSR, lysates | 2.0 ng/ml | <0.1 ng/m.l |
| BSR, supernatants | 1.9 ng/mi | <0.1 ng/ml |

These results show that RV-based vectors are able to efficiently produce HIV-1 Gag in human and non-human cell-lines and also confirm the previous finding that RV-based vectors are able to replicate efficiently in cells-lines derived from humans (31), an important requirement for an HIV-1 vaccine vector. These results were also confirmed on human and rhesus monkey PBMC.

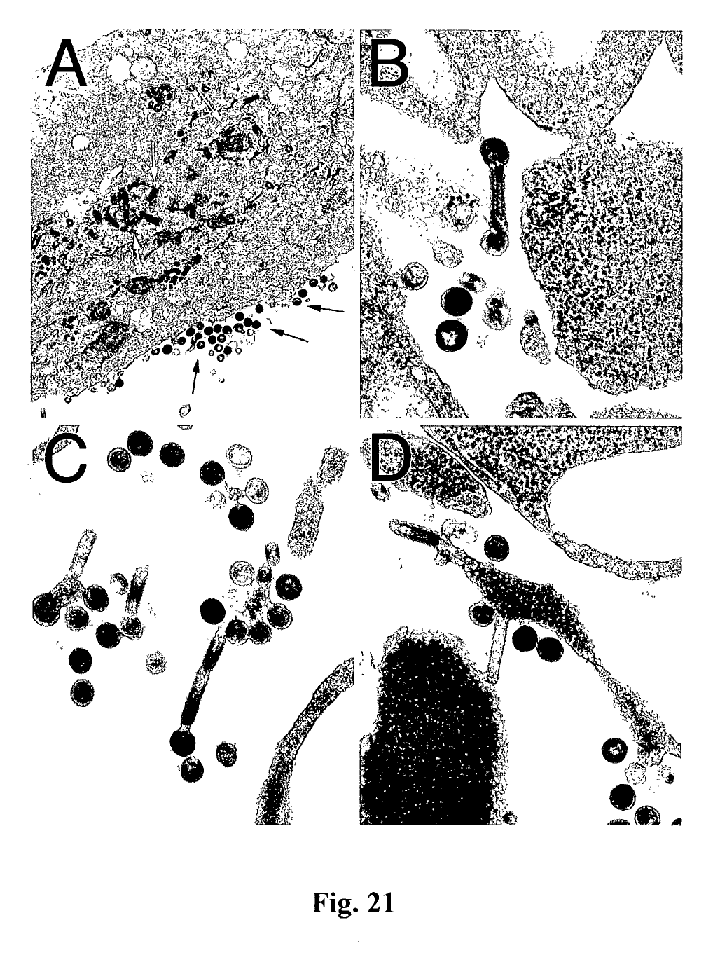

RV infection does not cause a cytopathic effect (CPE) on most cells lines, therefore the majority of the detected HIV-1 Gag was due to HIV-1 VLPs rather then free p55 from lysed cells. To study the generation of the HIV-1 VLPs, Hela cells were infected with an MOI of 1 for 48 hours. Cells were fixed at room temperature in neutral buffered 2.5% glutaraldehyde and gelled into warm agar. These cells were post-fixed in 1% OsO4, dehydrated in graded ethanol and propylene oxide, and embedded in Spurr's epoxy. Thin sections were cut and stained with uranly acetate and lead citrate and examined. The results shown in FIG. 21A indicated high production of RV (white arrows) and immature HIV-1 VLPs (black arrows), both on the plasma membrane and in cytoplasmic vacuoles. It is interesting to note that some HIV-l particles apparently budding from ends of RV particles (FIGS. 21B and 21C).

Induction of HIV-1 Gag-Specific CTL Responses in Mice Immunized with SPBN-Gag.

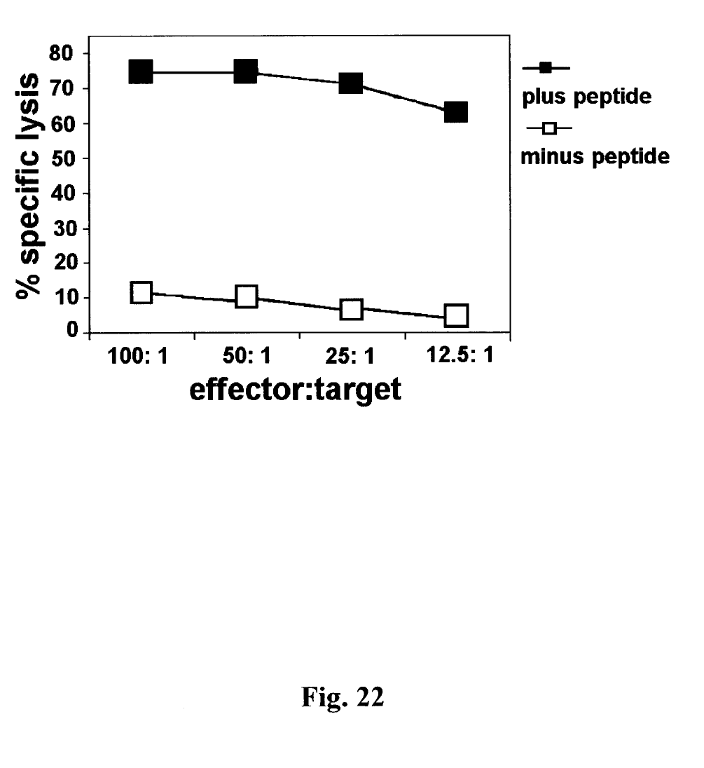

The recombinant RV expressing HIV-1 Gag was analyzed for the ability to induce a cellular immune response in mice. The results with recombinant RV expressing HIV-1 gp160 indicated that RV-based vectors were able to induce vigorous, long-lasting CTL responses after a single inoculation (supra). To analyze if this is the case for HIV-1 Gag, BALB/c mice were vaccinated once i.p with 10⁷ ffu of SPBN-Gag. Six weeks post-immunization, three mice were sacrificed and splenocytes were pooled and stimulated with naive mouse splenocytes that were pulsed with 10 μg/ml of MHC class I-restricted p24 peptide for seven days and cytolytic activity was measured by a standard chromium release assay (infra). As can been observed in FIG. 22, a strong cytotoxic response can only be detected against P815 target cells pulsed with p24 peptide and not against control cells without the p24 peptide. The same results were achieved in three independent experiments. Of note, the specific lysis still reached 70% at an effector to target ratio of 12.5:1, confirming that the priming with recombinant RVs is an excellent strategy for a potential HIV-1 vaccine. Because RV replicates efficiently in both human and non-human primate cells, similar responses would also be obtained in vaccinees other then mice. Even though the p24 peptide is MHC class I-restricted, conformation that the cytotoxic activity was mediated by CD8⁺ T-cells was performed. In vitro p24 re-stimulated splenocyte cultures of SPBN-Gag immunized mice were divided in CD8⁺/CD8⁻ cells. As expected, the CD8⁺⁻ depleted cultures showed no activity while the CD8⁺ T-cell enriched and unprocessed cultures showed high specific lysis. In addition, the CD8⁺ T-cell enriched population was also enriched in lytic units.

Flow Cytometry Analysis of IFN-γ Producing Cells.

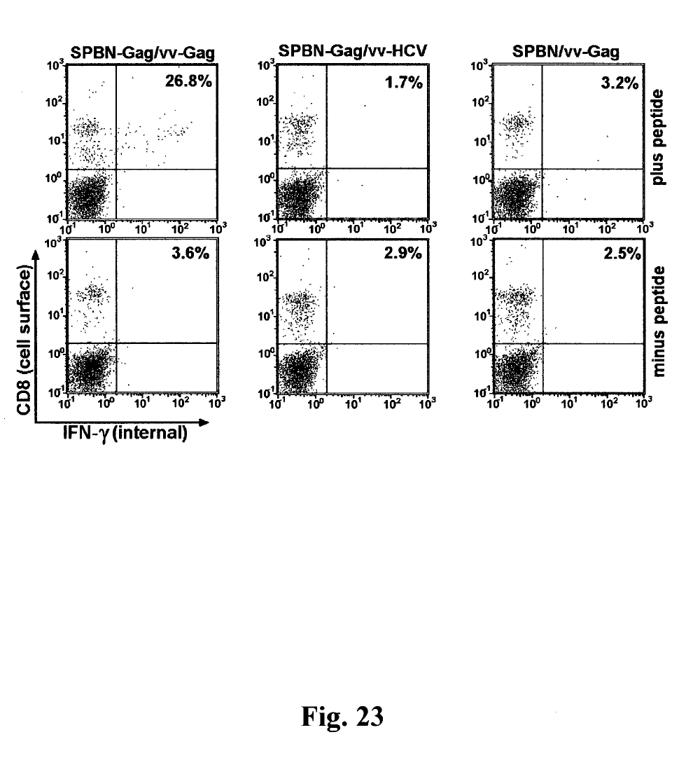

It has been shown clearly that CD8+ lymphocytes play an important role in controlling HIV-1 infection. The results described (supra) indicated that a RV-based vector expressing HIV-1 p55 is able to induce a potent, HIV-1 Gag-specific memory response, which was indicated by functional CTLs. In the next step, it was determined if successful priming with SPBN-Gag can also be detected in vivo. To analyze this in a more quantitative manners the percentage of IFN-γ CD8+ T cells were determined after challenge with a recombinant vaccinia virus expressing HIV-1 Gag (vv-Gag). Groups of ten BALB/c mice were immunized with SPBN-Gag or SPBN as a vector control. Nine weeks after immunization, mice were challenged with 10⁷ plaque forming units (pfu) of vv-Gag or a recombinant vaccinia virus expressing the structural proteins of HCV as a control (vv-HCV). Five days after the challenge infection, two mice in each group were sacrificed and spleens were removed. To determine the number of HIV-1 Gag-specific T cells expressing IFN-γ, splenocytes were cultured with or without p24 peptide for 24 hours followed by immunostaining with two antibodies directed against murine IFN-γ or CD8. The flow cytometry analysis is shown in FIG. 23. A high number of IFN-γ secreting cells (2.7% of the total splenocytes and 26.8% of the total CD8+ T-cells) were detected five days after the challenge with vv-Gag in spleens of SPBN-Gag immunized mice. As expected, control animals primed with SPBN and challenged with vv-Gag showed only a low number of IFN-γ secreting CD8+ T-cells, confirming that the high numbers of IFN-γ secreting CD8+ T-cells were due to the SPBN-Gag priming and not to the vv-Gag challenge.

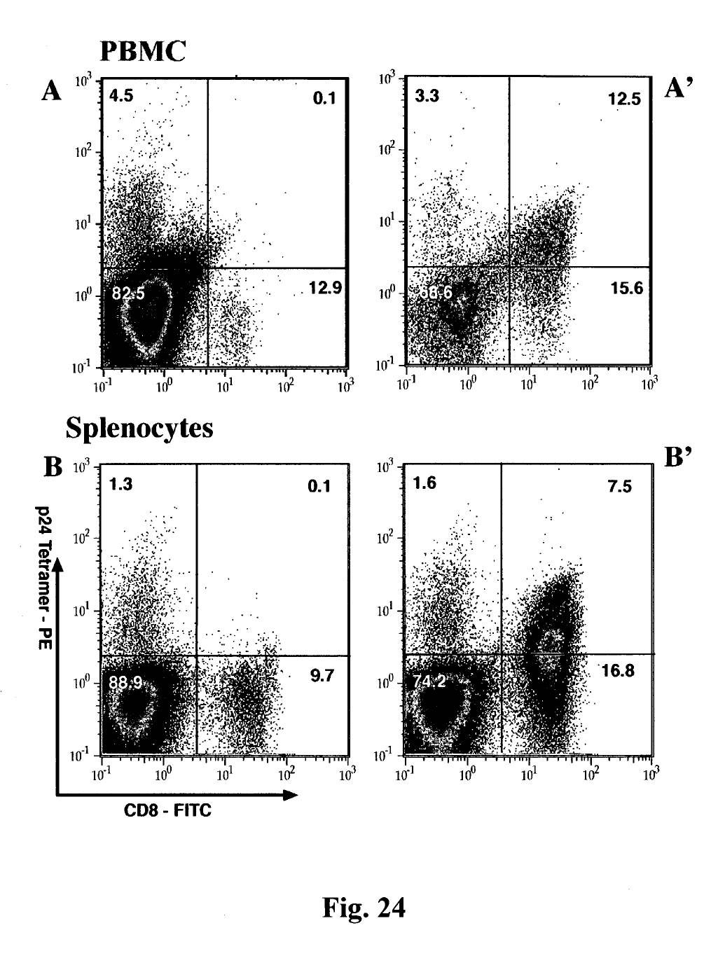

The high number of CD8+ T-cell after challenge with vv-Gag but not with vv-HCV was also confirmed by staining with the K^{d}-p24 tetramer specific for the immunodominant HIV-1 Gag epitope (AMQMLKETI) recognized in H-2d mice. 30.8% or 44.5% of the CD8+ T-cell from splenocytes or peripheral mononuclear cells (PBMCs) respectively, of SPBN-Gag immunized mice were teramer-positive after challenge with vv-Gag whereas only a low number (0.8%) of the CD8+ T cell were detected after the challenge with vv-HCV (FIG. 24).

Discussion

The present invention relates to RV-based vectors expressing HIV-1 envelope proteins. These vectors are able to induce a humoral response against HIV-1 gp160 after a single immunization followed by a boost injection with recombinant HIV-1 gp120. (Schnell, M. J., et al., Proc. Natl. Acad. Sci. USA, 97:3544-3549, 2000.). Expanding evidence suggests that CTL responses play a major role in the anti-viral immune response against HIV-1. (Brander, C. and B. D. Walker, Current Opinion in Immunology, 11:451-9, 1999.). The development of an effective prophylactic HIV-1 vaccine therefore requires the selection of HIV-1 antigen(s) capable of inducing long-lasting and broadly reactive CTL responses. The present invention further relates to RV-based vectors to induce such responses.

In contrast to the observed humoral response, a single inoculation of mice with a recombinant RV expressing HIV-1 envelope protein results in a vigorous CTL response against HIV-1 Env. In addition, these responses are stable for at least 135 days after immunization. One explanation for these strong responses is that RV grows in various cell-lines without killing the cells, which results in longer expression of HIV-1 genes compared to a cytopathogenic viral vector. In addition, the expression of the RV nucleoprotein, which was previously shown to be an exogenous superantigen (Lafon, M., Research in Immunology, 144:209-13, 1993; Lafon, M., et al., Nature, 358:507-10, 1992), might help to enhance a general immune response against the HIV-1 envelope after a single immunization.

The recombinant RVs of the present invention are able to induce cross-reactive CTLs against a variety of different HIV-1 envelope proteins. Previous studies showed that single amino acid exchanges can abrogate CTL cross-reactivity, whereas other examinations indicated that single or even double amino acid substitutions frequently did not abrogate cross-killing. (Cao, H., et al., J. Virol., 71:8615-23, 1997; Johnson, R. P., et al., Journal of Experimental Medicine, 175:961-71, 1992; Johnson, R. P., et al., Journal of Immunology, 147:1512-21, 1991.). Therefore, the question remains if CTLs induced by recombinant RVs are directed against different epitopes. However, several studies indicating that CTLs from HIV-1 infected individuals show cross-reactivity even with different clades of HIV-1, indicating a broad cross-reactivity, is an important requirement for an HIV-1 vaccine. (Cao, H., et al., J. Virol., 71:8615-23, 1997; Rowland-Jones, S. L., et al., Journal of Clinical Investigation, 102:1758-65, 1998.). There is currently only one study showing cross-clade CTLs reactivities induced with a canarypox-based HIV-1 vaccine in uninfected volunteers. (Ferrari, G., et al., Proc. Natl. Acad. Sci. USA, 1396-401, 1997.). The inventors of the present invention are currently analyzing if CTLs against HIV-1 gp160 induced by recombinant RV are also cross-reactive against HIV-1 envelope protein from clades other than B.

In summary, the present invention demonstrates the ability of the murine sera to neutralize HIV-1 strain. Thus the present invention shows that recombinant RVs are excellent vectors for B cell priming. The present invention also shows that a single vaccination with recombinant RV expressing HIV-1 envelope protein elicits a strong, long-lasting CTL response specific against HIV-1 proteins, such as the envelope protein of different HIV-1 strains. These results further emphasize the use of RV as an HIV-1 vaccine.

In contrast to most other viral vectors, only a negligible sero-positivity exists in the human population to RV and immunization with a RV-based vector against HIV-1 will not interfere with immunity against the vector itself. Because oral immunization against RV with a RV vaccine strain is successful and apathogenic in chimpanzees (Report of the forth WHO Consultion on oral immunization of dogs against rabies unpublished document WHO/Rab.Res./93.42, 1993.), a RV-based vector will also be promising in inducing mucosal immunity against HIV-1. In addition, it is possible to construct a RV expression vector that does not cause rabies even after direct inoculation into the brains of mice. Therefore, the present invention fulfills a long felt, yet unfulfilled need, for a method of treating HIV-1 infections. Using the recombinant RVs of the present invention, all of the dominant epitopes for neutralizing antibodies, cytotoxic lymphocytes, and antibody dependent cell cytotoxicity are expressed at one time, thereby eliciting both humoral and cell-mediated immunity against HIV-1.

In addition to the induction of both humoral and cellular immunity against HIV by the RV-based vaccine expression of the HIV-1 gp160, a strong cellular immune response was induced using an RV-based vaccine expression of the HIV-1 gag protein. The present invention discloses that RV-based vectors stably express HIV-1 Gag p55 precursor protein that is identical in size to Gag expressed by HIV-1. Infection of human cells with the recombinant RV expressing HIV-1 Gag resulted in a large quantity of immature HIV-1 VLPs in addition to bullet-shaped RV particles. The expression of HIV-1 Gag and production of VLPs has been shown previously with other expression systems, such as viral vectors and naked DNA (25, 43, 54). HIV-1 derived VLPs have been demonstrated to be immunogenic and are able to induce both cellular and humoral response (15, 51). One advantage of the RV-based system is that RV grows very efficiently on human cells without killing the infected cells (48, 53), thereby resulting in the long-term production of VLPs. Compared to DNA vaccines, the advantage of a replication-competent viral vector, such as RV, is the in vivo spread of the viral vector and therefore expression of the antigen in a large number of cells.

Research results indicated that Gag-specific CD8+ T-cells are important in controlling virus load during acute infection (27). In addition, recent research showed that multiple inoculation with a DNA vaccine encoding HIV-1 Gag and human IL-2 protected rhesus macaques from developing an AIDS-like disease (6). The present invention provides RV-based vectors expressing HIV-1 Gag for the protection against AIDS without the need for multiple inoculations. Another advantage of a Rhabdovirus-based vector is that, in contrast to DNA-based vaccines, no modification of the antigen encoding sequence(s) are required. Recent data indicate that numerous modifications, for example of HIV-1 gag, are required to achieve sufficient expression and immunogenicity in mice (39, 40, 54). Because RV exclusively replicates in the cytoplasm of the infected cells, expressing the Gag-encoding RNA is independent from the HIV-1 Rev and Rev responsive elements (RRE). This is also advantageous considering the dramatic variability of the HIV-1 genome, which may require vaccine vectors expressing an HIV-1 antigen from different strains or clades (1).

The present invention further relates to RV-based vectors expressing HCV envelope proteins. Currently, no method exists to propagate HCV in vitro (Frolov et al., 1999; Lohmann et al., 2001), which eliminates the possibility of utilizing attenuated or killed HCV as a vaccine strategy. The present invention provides HCV vaccines using both killed RV particles containing recombinant HCV E2 and live, replication-competent, RV vaccine strain-based vectors. Three RV vectors expressing HCV envelope proteins were constructed. One vector expresses the HCV envelope proteins E1 and E2. A second vector expresses a modified version of E2, with an 85 amino acid deletion at its carboxy-terminus, and the TMD and CD of human CD4. The third vector expresses the modified version of E2 with the TMD of human CD4 and the CD of the RV glycoprotein.

The protective immune response against HCV is not well-defined, the initial HCV vaccine approach was to focus on both arms of the immune response (i.e. humoral and cellular). Increasing evidence indicates that cellular immune responses play an important role for a self-limited HCV infections and recovery from HCV infection. In general, both CD4+ helper T-cells and CD8+ cytotoxic T-cells seem to be more frequent and stronger in patients who recover then patients that develop a chronic infection (Liang et al., 2000). Moreover, one study indicated that the number of IFN-γ producing cells during the first six months after the onset of disease is associated with eradication of the HCV infection (Gruener et al., 2000).

The present invention reveals that a RV vaccine vector is able to induce long-lasting CTL responses against HCV E2 but the specific killing was not as strong as previously seen for HIV-1 Gag or envelope (supra). Our data are consistent with those of other groups who used other HCV vaccine approaches in BALB/c mice and detected only a low percentage of specific CTLs against HCV E2 (Vidalin et al., 2000). More recently, Gordon et al. characterized a new MHC class I E2-specific epitope for the H-2d haplotype (Gordon et al., 2000), which may be helpful for further studies of cellular responses against HCV E2 in BALB/c mice. The present invention clearly indicates that RV-based vectors are potent vectors for the induction of E2-specific CTLs.

In contrast to the cellular response(s), the requirements for an HCV-specific humoral response for a HCV vaccine are more conflicting. Infection of host cells with enveloped viruses is typically mediated by an interaction between the viral glycoprotein(s) in the host-cell derived membrane and a cellular receptor(s) on the host cell. Previous studies indicate that the hypervariable region 1 (HVR1) of E2 binds to the cellular CD81 molecule of the host cell (Flint et al., 1999). Hence, it is probable that host-produced antibodies against E2 would neutralize the attachment and/or fusion of HCV virions to host cells during a natural infection.

The present invention provides a new vaccine strategy to immunize against HCV. Killed RV particles containing HCV E2 proteins are able to induce vigorous B-cell responses (supra). The reason for these strong responses could be that a viral glycoprotein displayed on a viral particle is more immunogenic than its soluble form. It has been shown for RV that soluble G, in contrast to the virion-associated G, fails to protect from lethal RV challenge (Dietzschold et al., 1983).

In summary, the present invention provides HCV proteins that are stably expressed and induce a long-lasting cellular response as well as a strong E2-specific B-cell response in vivo.

### EXAMPLES

The following examples further illustrate the present invention, but of course are not in any way limiting its scope. The examples below are carried out using standard techniques, that are well known and routine to those of skill in the art, except where otherwise described in detail. The examples are illustrative, but do not limit the invention. All animal methods of treatment or prevention described herein are preferably applied to mammals, most preferably to humans.

### Example 1

### Plasmid Construction

HIV Constructs

Shown in FIG. 1 is a schematic representation of a method for the construction of recombinant RV genomes. At the top, the wild-type RV genome with its five open reading frames is shown (SAD L16). Using a PCR strategy and site directed mutagenesis the entire T gene is removed and a new minimal RV transcription unit containing two single sites is introduced between the G and L genes (SBN). The cDNA sequence encoding HIV-1_{89.6} or HIV-1_{NL4-3} gp160 is inserted using the BsiWI and NheI sites resulting in the plasmids, pSBN-89.6 or pSBN-NL4-3 (bottom).

Two single sites are introduced in the previously described RV cDNA pSAD L16 upstream of the G (SmaI) and ψ gene (NheI) by site directed mutagenesis (GeneEditor™ Promega Inc.) using the primers RP11

| | |
|---|---|
| RP11 | (SEQ ID NO: 1) |

5′-CCTCAAAAGACCCCGGGAAAGATGGTTCCTCAG-3′ and

| | |
|---|---|
| RP12 | (SEQ ID NO: 2) |

5′-GACTGTAAGGACYGGCTAGCCTTTCAACGATCCAAG-3′

resulting in the plasmid pSN. pSN is the target used to introduce a new transcription Stop/Start sequence, as well as a single BsiWI site using a polymerase chain reaction (PCR) strategy. First, two fragments are amplified by PCR from pSN using Vent polymerase (New England Biolabs Inc.) and the forward primers RP1

| | |
|---|---|
| RP1 | |
| 5′-TTTTGCTAGCTTATAAAGTGCTGGGTCATCTAAGC-3′ or | (SEQ ID NO: 3) |
| RP10 | |
| 5′-CACTACAAGTCAGTCGAGACTTGGAATGAGATC-3′. | (SEQ ID NO: 4) |
| The reverse primers were RP18 | |
| 5′-TCTCGAGTGTTCTCTCTCCAACAA-3′ and | (SEQ ID NO: 5) |
| RP17 | |
| 5′-AAGCTAGCAAAACGTACGGGAGGGGTGTTAGTTTTTTTCATGGACTTGGATCGTTGAAAGGACG-3′. | (SEQ ID NO: 6) |

RP17 contains a RV transcription Stop/Start sequence (underlined) and a BsiWI and NheI site (shown in italics). PCR products are digested with NheI, ligated, and the 3.5 kb band eluted from an agarose gel. After gel elution the band is digested with ClaI/MluI and ligated to the previously Clal/Mlul digested pSN. The plasmid is designated pSBN.

The HIV-1 gp160 genes, encoding the envelope protein of the HIV-1 strains 89.6 and NL4-3, are amplified by PCR using Vent polymerase, the forward primer 5′-GGGCTGCAGCTCGAGCGTACGAAAATGAGAGTGAAGGAGATCAGG-3′ (SEQ ID NO: 7) containing PstI/Xhol/BsiWI sites (italics), and the reverse primer 5′-CCTCTAGATTATAGCAAAGCCCTTTCCAAG-3′ (SEQ ID NO: 8) containing a XbaI (italics) site. The PCR products are digested with PstI and XbaI and cloned to pBluescript II SK+ (Stratagene). After conformation of the sequence, the HIV-1 gp160 genes are excised with BsiWI and XbaI and ligated to pSBN, which had been digested with BsiWI and NheI. The resulting plasmids are entitled pSBN-89.6 and pSBN-NL4-3.

pSBN was described previously (47) and was the target to introduce a new single restriction site (PacI, bold) downstream of the RV G gene by site-directed mutagenesis (GeneEditor) using the and the primer 5′-GTGAGACCAGACTGTAATTAATTAACGTCCTTTCAACGATCC-3′ (SEQ. ID. NO: 19) as indicated by the manufacturer (Promega). The resulting plasmid was designated pSPBN. The gene encoding HIV-1_{NL4-3} gag was amplified by PCR from pNL4-3 (3) using Vent DNA polymerase (New England Biolabs) and the forward primer RPx 5′-AAACTCGAGCGTACGACAATGGGTGCGAGAGCGTCAG-3′ (SEQ. ID. NO: 20) containing a BsiWI site (bold), and the reverse primer RPx 5′-AAAGCTAGCTTAATTAATCGCGATTATTGTGACGAGGGGTCG-3′ (SEQ. ID. NO: 21) containing a NheI (bold) site. The PCR product was digested with BsiWI and NheI and cloned to pSPBN, which had been previously digested with BsiWI and NheI. The resulting plasmid was entitled pSPBN-Gag.

HCV Constructs

All polymerase chain reactions (PCR) were performed using high fidelity Vent DNA polymerase (New England Biolabs) to minimize the introduction of sequence errors. pSBN was described previously (Schnell et al., 2000) and was the target to introduce a new single restriction site (PacI, bold) downstream of the RV G gene by site-directed mutagenesis (GeneEditor) using the primer 5′-GTGAGACCAGACTGTAATTAATTAACGTCCTTTCAACGATCC-3′ (SEQ. ID. NO: 9), as indicated by the manufacturer (Promega). The resulting plasmid was designated pSPBN. The gene encoding the structural proteins E1E2p7 of HCV was amplified by, PCR from pTM1/E1E2p7 (Michalak et al., 1997), using the forward primer RP58 5′-CTCGAGCGTACGAAAATGAATTCCGACCTCATGG-3′ (SEQ. ID. NO: 10) containing a BsiWI site (bold), and the reverse primer RP59 5′-CGTTAAGCTAGCTCATGCGTATGCCCGCTG-3′ (SEQ. ID. NO: 11) containing a NheI (bold) site. The PCR product was digested with BsiWI and NheI and cloned into pSPBN previously digested with BsiWI and NheI. The resulting plasmid was entitled pSPBN-E1E2p7. A recombinant RV expressing the ectodomain (ED) of HCV E2, with an 85 amino acid deletion at its carboxy-terminus, fused to the transmembrane domain (TMD) and cytoplasmic domain (CD) of human CD4, was amplified by PCR from pTM1/E2₆₆₁-CD4 (Cocquerel et al., 1998) using the forward primer RP 5′-CTCGAGCGTACGAAAATGGTCCTGGTAGTGCTG-3′ (SEQ. ID. NO: 12) containing a BsiWI site (bold), and the reverse primer RP 75 5′AATTGCTAGCTCAAATGGGGCTACATGTCTTC-3′ (SEQ. ID. NO: 13) containing a Nhe site (bold). The PCR product was cloned into pSPBN using the unique BsiWI and NheI sites resulting in pSPBN-E2CD4.

To construct a RV encoding the HCV E2 ED with an 85 amino acid deletion at its carboxy-terminus and containing the CD4 TMD and the RV G CD (rather than the CD4 CD) was PCR amplified from pSBN (Schnell et al., 2000) using the forward primer RP29 5′-CCC GGGTTAACAGAAGAGTCAATC GATCAGAAC-3′ (HpaI, bold; SEQ. ID. NO: 14) and the reverse primer RP8 5′-CCTCTAGATTACAGTCTGGTCTCACCCCC-3′ (XbaI, bold; SEQ. ID. NO: 15). The ED of HCV E2, with an 85 amino acid deletion at its carboxy-terminal end, fused to the TMD of CD4 was amplified by PCR from pTM1/E21₆₆₁-CD4 using the primers RP74 and RP57 5′-AACGAAGAAGATGCCTAGCCC-3′ (SEQ. ID. NO: 16). The first PCR product was digested with Hpal, ligated to the second one and the ligation was PCR re-amplified with the primers RP56 and RP8. The PCR product was cloned utilizing the BsiWI and XbaI sites into pSPBN previously digested with BsiWI and NheI. The resulting plasmid was designated pSPBN-E2CD4G.

### Example 2

### Recovery of Infectious RV from cDNA

For rescue experiments of the recombinant RVs, the previously described vaccinia virus-free RV recovery system is used (see Finke, et al., Journal of Virology, 73:3818-25, 1999). In brief, BSR-T7 cells, which stably express T7 RNA polymerase (a generous gift of Drs. S. Finke and K.-K. Conzelmann) are transfected with 5 μg of full-length RV cDNA in addition to plasmids coding for the RV N-, P-, and L-proteins (2.5 μg, 1.25 μg, and 1.25 μg) respectively, using a Ca₂PO₄ transfection kit (Stratagene) as indicated by the vendor. Three days after transfection, tissue culture supernatants are transferred onto fresh BSR cells and infectious RV is detected three days later by immunostaining against the RV-N protein (Centocor).

### Example 3

### One-Step Growth Curve

Shown in FIG. 2 is a graph showing One-step growth curves of recombinant RV BSR cells that are infected with the recombinant RVs (SBN, SBN-89.6, and SBN-NL4-3). The viral titers are determined in duplicate at the indicated time-points.

BSR cells (a BHK-21 clone) are plated in 60 mm dishes and 16 hours later infected (7×10₆ cells) with a multiplicity of infection (MOI) of 5 with SBN, SBN-89.6, or SBN-NL4-3 in a total volume of 2 ml. After incubation at 37° C. for 1 hour, inocula are removed and cells are washed four times with phosphate-buffered saline (PBS) to remove any unabsorbed virus. Three milliliters of complete medium is added back and 100 μl of tissue culture supernatants are removed at 4, 16, 24 and 48 hours after infection. Virus aliquots are titered in duplicate on BSR cells.

In FIG. 3 the Western blot analysis of recombinant RVs expressing HIV-1, gp160 is shown. Sup-T1 cells are infected with a MOI of 2 with SBN, SBN-89.6, or SBN-NL4-3 and lysed 24 h later. Proteins are separated by SDS-PAGE and analyzed by Western blotting. An antibody directed against gp120 detected two bands at the expected size for HIV-1 gp160 and gp120 in cell-lysates infected with SBN-89.6 or SBN-NL4-3 (α-gp120, lanes 3 and 4). No signal is detected either in the mock or SBN infected cells ((a-gp120, lanes 1 and 2). Successful infection of the cells by the recombinant RVs is confirmed with a polyclonal antibody directed against RV ((a-rabies, lanes 2, 3, and 4).

Shown in FIG. 4. are Sup-Ti cells which are infected using a MOI of 1 with SBN, SBN-89.6, or SBN-NL4-3. Twenty-four hours after infection, syncytia-formation is detected in cell cultures infected with recombinant RV expressing HIV-1 gp160 (panel SBN-89.6 and SBN-NL4-3), indicating expression of functional HIV-1 envelope protein. No cell fusion is detected in cultures infected with wild-type RV (panel SBN).

### Example 4

### Immunization

HIV gp120 Immunization

Groups of five 4-6 week old female BALB/c mice obtained from Jackson Laboratories are inoculated subcutaneously in both rear footpads with 10⁶ foci forming units (FFU) SBN, SBN-89.6, or 10⁵ NL4-3 in DMEM+10% FBS. Three out of five mice in each group are boost immunized intraperitonealy three months after infection with 10 μg recombinant gp41 (IIIB, Intracel Inc.) and 10 μg recombinant gp120 (IIIM, Intracel Inc.) in 100 μl complete Freunds adjuvant.

HIV Gag Immunization

Groups of five 6-8 week old female BALB/c mice (Harlan) were inoculated intraperitoneally (i.p) with 10⁷ foci-forming units (ffu) of SPBN-Gag. To analyze the induction of specific CTL response against HIV-1 Gag, spleens (infra) from two mice of each group were aseptically removed, combined and single cells suspensions were prepared. Red blood cells were lysed with ACK lysing buffer (BioWhitaker), splenocytes washed twice in RPMI-10 media containing 10% fetal bovine serum and pulsed with 10 μg/ml of the MCH class-I-restricted p24 peptide [aa AMQMLKETI; SEQ. ID. NO: X] and 10% T-STIM (Collaborative Biomedical Products) was added as a source of interleukin-2 (IL-2). Cytolytic activity of cultured CTLs was measured by a 4-hr assay with ⁵¹Cr labeled P815 target cells (infra).

HCV Immunization for Humoral Response

To analyze seroconversion against HCV E2, mice were immunized intraperitoneally (i.p.) with 1×10⁷ FFU of the respective RV. For vaccination with killed RV particles, sucrose purified RV (SPBN or SPBN-E2CD4G) was deactivated by incubation with β-Propiolactone (1:1000) overnight at 4° C. followed by another incubation at 37° C. for 30 minutes. Mice were vaccinated/boosted i.p. with 20 μg of killed RV particles as indicated in the text and figure legends. Ten days after the boost, sera were collected and analyzed for HCV-specific antibodies by ELISAs (infra).

HCV Cytotoxiciy Assays for CTL Response

Groups of five 6 to 8 week old female BALB/c mice (Harlan) were inoculated intraperitoneally (i.p.) with 10⁷ foci-forming units (FFU) of SPBN-E1E2p7. To analyze the induction of specific CTL response against E2, spleens from three mice of each group were aseptically removed, combined, and single cells suspensions were prepared. Red blood cells were lysed with ACK lysing buffer (BioWhitaker), splenocytes washed twice in RPMI-1640 media containing 10% fetal bovine serum and pulsed with 5 μg/ml peptide1323 [EATYSRCGSGPWITPRCMVD (SEQ. ID. NO: 17), amino acids 592-610 in HCV strain 1a] and 10% T-STIM (Collaborative Biomedical Products) was added as a source of interleukin-2 (IL-2). Cytolytic activity of cultured CTLs was measured by a 4-hour assay with ⁵¹Cr labeled P815 target cells. Target cells were prepared by incubating with 10 μg/ml peptide1323 and 100 μCi ⁵¹Cr for two hours and washed twice. Target cells were added to effector cells at various E:T ratios for four hours. The percent specific ⁵¹Cr release was calculated as 100×(experimental release−spontaneous release)/(maximum release−spontaneous release). Maximum release was determined from supernatants of cells that were lysed by the addition of 5% Triton X-100. Spontaneous release was determined from target cells incubated without added effector cells.

### Example 5

### Enzyme-Linked Immunosorbent Assay (ELISA)

HIV gp120 Assay

Recombinant HIV-1 gp120 (IIIB strain, Intracel) is resuspended in coating buffer (50 mM Na₂CO₃, pH 9.6) at a concentration of 200 ng/ml and plated in 96 well ELISA MaxiSorp plates (Nunc) at 100 μl in each well. After overnight incubation at 4° C., plates are washed three times (PBS pH 7.4, 0.1% Tween-20), blocked with blocking buffer (PBS, pH 7.4, 5% dry milk powder) for 30 minutes at room temperature, and incubated with serial dilutions of sera for 1 hour. Plates are washed three times followed by the addition of horseradish peroxidase-conjugated (HRP) goat anti-mouse-IgG (H+L) secondary antibody (1:5000, Jackson ImmunoResearch Laboratories). After a 30 minute incubation at 37° C., plates are washed three times and 200 μl OPD-substrate (o-phenylenediamine dihydrochloride, Sigma) is added to each well. The reaction is stopped by the addition of 50 μl of 3 M H₂SO₄ per well. Optical density is determined at 490 nm. Shown in FIG. 5 is a graph depicting ELISA reactivity of mouse sera against HIV-1 gp120. Five mice each are immunized with recombinant RVs (SBN, SBN-89.6, or SBN-NL4-3) and 3 months after the initial infection three mice from each group are boosted with recombinant HIV-1 gp120 and gp41(SBN*, SBN-89.6*, or SBN-NL4-3*). Each data point on the graph indicates the average of mice from each group in three independent experiments. One mouse of the SBN-89.6 group did not react to the boost injection and is not included in the graph. The error bars indicate the standard deviations.

HIV Gag Assay

Hela cells were infected with a MOI of 5. 48 hours later, supernatants were collected and cells resuspended in Lysing Buffer (Triton X-100 in PBS and 2-chloroacetamide). The supernatants and cell suspension were transferred to microcentrifuge tubes and spun for two minutes at 14,000 rpm to remove cell debris. The quantification of Gag p24 protein in cell supernatants and lysates was performed using the p24 antigen enzyme-linked immunosorbent assay (ELISA), as described by the manufacturer (ZeptoMetrix, Inc.).

HCV Assay

96-well MaxiSorp plates (Nunc) were coated with recombinant E2 (ImmunoDiagnostic Inc.) in coating buffer (50 mM Na₂CO₃ pH 9.6) at a concentration of 2.5 μg/ml and incubated overnight at 4° C. Plates were washed three with 0.05% PBS/Tween and blocked with 5% dry milk powder in PBS for one hour at room temperature. Mouse sera were diluted in 1X PBS, added to the plates and incubated at room temperature for one hour. After washing three times with 0.05% PBS/Tween, the secondary antibody (goat a-mouse HRP conjugated, Jackson ImmunoResearch) diluted 1:5000 in 1X PBS was added and the plates were incubated for 30 minutes at 37° C. OPD substrate (Sigma) was added to the plates after washing three times with 0.05% PBS/Tween. Substrate reaction was stopped by the addition of 50 μl 2M H₂SO₄ to each well. Plates were read at 490 nm.

### Example 6

### Western Blotting

HIV

Human T-lymphocytic cells (Sup-T1) cells are infected with a MOI of 2 for 24 hours and resuspended in lysis buffer 50 mM Tris, pH 7.4; 150 mM NaCl, 1% NP-40, 0.1% SDS, and 1× protease inhibitor cocktail (Sigma) for 5 minutes. The protein suspension is transferred to a microfuge tube and spun for 1 minute at 10,000×g to remove cell debris. Proteins are separated by 10% SDS-PAGE and transferred to a PVDF-Plus membrane (Osmonics). After blocking for 1 hour (5% dry milk powder in PBS pH 7.4), blots are incubated with sheep a-gp120 antibody (ARRRP) (1:1000) or human a-rabies sera (1:500) in blocking buffer for 1 hour. Secondary antibodies of goat α-human or donkey α-sheep horseradish peroxidase-conjugated (HRP) antibodies (1:5000) (Jackson ImmunoResearch Laboratories) are added and blots incubated for one hour. Each antibody incubation is followed by three washes with WB-wash buffer (PBS pH 7.4, 0.1% Tween-20). Chemiluminescence (NEN) is performed, as directed by the manufacturer.

Western blot analysis to detect anti-HIV-1 antibody is performed using a commercial Western Blot kit (QualiCode HIV-1/2 Kit, Immunetics) according to the manufacturer's instructions, except for the mouse sera in which a-human IgG conjugate is substituted with a 1:5000 dilution of an alkaline phosphatase-conjugated goat anti-mouse IgG (H+L) (Jackson ImmunoResearch Laboratories). Shown in FIG. 6 is the Western blot analysis of mice serum antibody response to HIV-1 antigens. Sera from one mouse of each group (SBN, SBN-89.6, or SBN-NL4-3), which are immunized by the RVs (α-SBN, α-SBN-89.6 or α-SBN-NL4-3), or immunized and boost injected with recombinant gp120 and gp41(α-SBN*, α-SBN-89.6* or α-SBN-NL4-3*), are tested at 1:100 dilutions. A highly positive and weakly positive human control serum is used to detect the position of the HIV-1 proteins. SC indicates the serum control.

HCV

BSR cells were infected with a MOI of 5 for 48 hours and resuspended in lysis buffer [50 mM Tris, pH 7.4/150 mM NaCl/1% NP-40/0.1% SDS/1X protease inhibitor cocktail (Sigma)] on ice for five minutes. The suspension was transferred to a microcentrifuge tube and spun for one minute at 14,000 rpm to remove cell debris. Proteins were separated by 10% SDS/PAGE and transferred to a PVDF-Plus membrane (Osmonics, Minnetonka, Minn.). Blots were blocked for one hour [5% dry milk powder in PBS (pH 7.4)]. After blocking, blots were washed twice using a 0.1% PBS-Tween-20 solution and incubated with either monoclonal murine α-E2 antibody (H52, 1:1000) (Flint et al., 1999), monoclonal murine α-E1 antibody (A4, 1:1000) (Dubuisson, 2000) or rabbit α-RV-G tail antibody (1:20,000) (Foley et al., 2000) in 0.1% PBS-Tween for one hour. Blots were then washed three times with 0.1% PBS-Tween. Secondary antibodies of goat a-mouse or donkey a-rabbit HRP conjugated antibodies (1:5,000) (Jackson ImmunoResearch) were added, and blots were incubated for 1 hour. Again, blots were washed three times with 0.1% PBS-Tween and washed once with PBS (pH 7.4). Chemiluminescence (NEN) was performed as instructed by manufacturer. For quantification, Hyperfilm ECL film (Amersham Pharmacia Biotech) was preflashed with a Sensitize Preflash Unit as indicated by the manufacturer (Amersham Pharmacia Biotech) scanned and quantification was performed with NIH Image, version 1.61.

### Example 7

### Virus Neutralization Assays

HIV-1 strains are recovered on 293T cells and virus stocks are expanded on MT-2 cells (HIV-1 NL4-3), frozen at −75° C. and titered on MT-2 cells. Neutralization assays are performed according to Montefiori et al., (Journal of Clinical Microbiology, 26, 231-5, 1998). Briefly, ˜5000 TCID₅₀ of HIV-1_{NL4-3} are incubated with serial dilutions of mouse sera for 1 hour. MT-2 cells are added and incubated at 37° C., 5% CO₂ for 4-5 days. 100 μl of cells are transferred to a poly-L-lysine plate and stained with neutral red dye (Neutral Red, ICN) for 75 minutes. Cells are washed with PBS, lysed with acid alcohol and analyzed using a colorimeter at 550 nm. Protection is estimated to be at least 50% virus inhibition.

### Example 8

### Preparation of Splenocytes

Spleens are aseptically removed and single cells suspensions are prepared. Red blood cells are lysed with ACK lysing buffer (BioWhitaker) and the remaining splenocytes are washed twice in RPMI-10 media containing 10% fetal bovine serum. Splenocytes are divided into effector and stimulator cells. Stimulator cells are prepared by infection with recombinant vaccinia virus (moi=10) expressing an envelope protein from HIV-1 at a multiplicity of infection (moi) of 1 for two hours. Cells are washed with PBS once to remove excess virus and incubated for 16 hours at 37° C. After incubation, the vaccinia virus is inactivated using Psoralen (Sigma) (infra). Stimulator cells are added back to the effector cell population at a ratio of 3:1 and 10% T-STIM (Collaborative Biomedical Products) is added as a source of interleukin-II (IL-2).

### Example 9

### Inactivation of Virus with Psoralen

Following incubation of splenocytes with the vaccinia virus, the virus is inactivated using psoralen (Sigma). Psoralen is added to cells to achieve a final concentration of 5 μg/ml. Following a ten minute incubation at 37° C. the cells were treated with long-wave UV (365 nm) for 4 minutes and washed twice with PBS.

### Example 10

### Preparation of Chromium Labeled Target Cells

HIV gp160 CTL Response

Target cells (P815) are prepared by infection with vaccinia virus expressing the HIV-1 protein (see specific figure legend for specific protein) for one hour at a moi of 10, washed to remove excess virus, and incubated for 16 hours at 37° C. To measure background, target cells are infected with vaccinia virus expressing HIV-1 Gag (vP1287) or wild-type vaccinia (vP1170). Target cells are washed once in PBS, incubated with 100 μCi ⁵¹Cr for one hour to label the cells, washed two times in PBS and added to effector cells at various E:T ratios (see figures) for four hours at 37° C. The percent specific ⁵¹Cr release is calculated as 100×(experimental release−spontaneous release)/(maximum release−spontaneous release). Maximum release was determined from supernatants of cells that were lysed by the addition of 5% Triton X-100. Spontaneous release was determined from target cells incubated without added effector cells.

HIV Gag CTL Response

Target cells were prepared by incubating with 10 μg/ml p24 peptide and 100 μCi ⁵¹Cr for two hours and washed twice. Target cells were added to effector cells at various E:T ratios for four hours. The percent specific ⁵¹Cr release was calculated as 100×(experimental release−spontaneous release)/(maximum release−spontaneous release). Maximum release was determined from supernatants of cells that were lysed by the addition of 5% Triton X-100. Spontaneous release was determined from target cells incubated without added effector cells.

### Example 1

### Preparation of CD8+ Depleted T Cells

Seven days post in-vitro stimulation, CD8⁺ T-cells are depleted from the cell culture (CD8⁻) and enriched (CD8⁺) using Dynabeads Mouse CD8 (Lyt2), as described by the manufacturer.

### Example 12

### Immunofluorescence Microscopy

HIV-1 Gag Experiments

BSR or HeLa cells were plated in 6-well plates containing coverslips and infected with a multiplicity of infection (MOI) of 0.1 for 48 hours. Cells were fixed with 80% acetone at 4° C. for 30 minutes. Cells were incubated with a monoclonal human antibody directed against HIV-1 p24 antigen (20) (1:50) for 1 hour at room temperature and again washed three times with PBS. After incubation for another 30 min with goat anti-human FITC 1:200 (Jackson ImmunoReasearch), cells were washed three times with PBS and analyzed by fluorescent microscopy. A FITC-labeled antibody against RV N (Centocor) was used as described previously (18, 47).

HCV Experiments

BSR cells were plated in six-well plates containing coverslips and infected with a multiplicity of infection (MOI) of 0.1 for 48 hours. Cells were fixed with 4% paraformaldehyde at room temperature for 20 minutes. For internal immunostaining cells were permeabilized with 1% Triton in (phosphor-buffered saline) PBS for 5 minutes at room temperature. Cells were washed three times with PBS-Glycine [10 mM glycine in PBS (pH 7.4)] and incubated with a monoclonal mouse antibody directed against HCV E2 (H53, 1:600) for 1 hour at room temperature and again washed three times with PBS-Glycine. After incubation for another 30 min with donkey anti-mouse FITC 1:100 (Jackson ImmunoReasearch) cells were washed three times with PBS-Glycine and analyzed by fluorescence microscopy. A FITC-labeled antibody against RV N (Centocor) was used as described previously (Foley et al., 2000; Schnell et al., 2000).

### Example 13

### Use of E2 Proteins Derived from Purified Recombinant Virions

Recombinant RVs in the supernatants from SPBN or SPBN-E2CD4RVG infected BSR cells were sucrose purified and incubated for 30 minutes with 1% Triton X-100 in PBS. RV Ribonucleoprotein (RNP) complex was removed by centrifugation at 16,000 g at 4° C. for an hour. Supernatants were removed and used directly to coat ELISA plates or frozen at 80° C. 96-well MaxiSorp plates (Nunc) were coated with glycoprotein(s) derived from 25 μg purified SPBN or SPBN-E2CD4G virions for each plate in coating buffer (50 mM Na₂CO₃, pH 9.6) and incubated overnight at 4° C. Plates were washed three times with 0.05% PBS/Tween and blocked with 5% dry milk powder in 1X PBS for 1 hour at room temperature. Human sera were diluted in 1X PBS beginning with a 1:100 dilution, added to the plates, and incubated at room temperature for 1 hour. Plates were washed three times with 0.05% PBS/Tween and the secondary antibody (goat a-human horse radish peroxidase (HRP) conjugated, Jackson ImmunoResearch) diluted 1:5000 in PBS was applied and plates were incubated at 37° C. for 30 minutes. Plates were washed three times with 0.05% PBS/Tween, and OPD substrate (Sigma) was added as instructed by the vendor. Substrate reaction was stopped by the addition of 50 μl 2 M H₂SO₄ to each well. Plates were read at 490 nm using a Bio-Tek ELₓ800 plate reader.

### Example 14

Electron Microscopy of Recombinant RV Expressing HIV-1 Gag

HeLa cells were infected with a MOI of 1 for 48 hours. Cells were fixed at room temperature in neutral buffered 2.5% glutaraldehyde and gelled into warm agar. They were post-fixed in 1% OsO4, dehydrated in graded ethanol and propylene oxide, and embedded in Spurr's epoxy. Thin sections were cut and stained with uranly acetate and lead citrate and examined with a LEO EM 10 electron microscope at 60 kV.

### Example 15

Intracellular INF-γ Staining of CD8⁺ T-Cells and Flow Cytometry Analysis.

Groups of 6- to 8-week-old female Balb/c mice were inoculated i.p. with 10⁷ ffu of recombinant RV expressing HIV-1 Gag (SPBN-gag) or vector alone (SPBN). Nine weeks post-immunization, mice were challenged with vaccinia virus expressing HIV-1 gag ((23), vP1287) or an unrelated hepatitis C virus (HCV) protein. Five days later, spleens were removed, single cell suspensions were prepared (supra) and red blood cells removed. Cells were cultured at 2×10⁶ cells/ml with or without p24 peptide (AMQMLKETI; SEQ. ID. NO: 18) for 16 hours, and treated with GolgiPlug (Pharminogen) to inhibit cytokine secretion from the Golgi apparatus for an additional 6 hours. Cells were incubated with rat anti-mouse CD16/CD32 (1 μg/10⁶ cells) (Pharminogen) to inhibit non-specific binding by the fluorescent antibodies. Cells were washed twice with Staining Buffer (PBS containing 1% FBS), then stained with PE-conjugated monoclonal rat anti-mouse CD8 antibody (0.06 g/10⁶ cells) (Pharminogen) and washed twice in staining buffer. Cells were resupended in 50 μl Staining Buffer, 100 μl Fixation Medium (Caltag Laboratories) was added and incubated for 15 minutes, room temperature. Fixed cells were washed three times with PBS and resuspended in 100 μl Permeabilization Medium (Caltag Laboratories) containing 0.12 μg/10⁶ cells FITC-conjugated rat anti-mouse IFN-γ monoclonal antibody (Phaminogen) for 15 minutes, room temperature. Cells were washed three times with PBS and counted by flow cytometry. Surface (IgG2a K) and internal (IgG1) isotype-matched negative controls (Pharminogen) were analyzed as control samples.

All publications and references, including but not limited to patent applications, cited in this specification, are herein incorporated by reference in their entirety as if each individiual publication or reference were specifically and individually indicated to be incorporated by reference herein as being fully set forth.

While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. A recombinant Rhabdovirus vector comprising:
a) a modified Rhabdovirus genome;
b) a new transcription unit inserted into the Rhabdovirus genome to express heterologous nucleic acid sequences; and
c) a heterologous viral nucleic acid sequence that is inserted into said new transcription unit,
wherein the recombinant Rhabdovirus vector is replication competent, and said heterologous viral nucleic acid sequence encodes an antigenic polypeptide.

2. The recombinant Rhabdovirus vector of claim 1, wherein said modified rhadovirus genome is a modified rabies virus genome.

3. The recombinant Rhabdovirus vector of claim 2, wherein said modified rabies virus genome has a second modification to have a glycoprotein from another class of virus in place of a rabies virus glycoprotein.

4. The recombinant Rhabdovirus vector of claim 3, wherein said glycoprotein from another class of virus is vesicular stomatitis virus glycoprotein.

5. The recombinant Rhabdovirus vector of claim 3, wherein said modified rabies virus genome has a third modification to have contiguity of structural genes different from that in said modified rhabodvirus genome after said second modification.

6. The recombinant Rhabdovirus vector of claim 1, wherein said heterologous viral nucleic acid encodes an antigenic polypeptide selected from the group consisting of a full-length HIV envelope protein, HIV gp160, HIV gag, HIV gp120, and full-length SIV envelope protein.

7. The recombinant Rhabdovirus vector of claim 6, wherein said heterologous viral nucleic acid is fused to a sequence of a cytoplasmic domain of a glycoprotein gene of said modified Rhabdovirus genome to produce a chimeric protein such that said chimeric protein has a fusion between a transmembrane domain of said heterologous protein and a cytoplasmic domain of said glycoprotein.

8. The recombinant Rhabdovirus vector of claim 1 further comprising a deletion of a recombinant Rhabdovirus glycoprotein gene, and wherein said heterologous viral nucleic acid is fused to a sequence of a cytoplasmic domain of a glycoprotein gene of said modified Rhabdovirus genome to produce a chimeric protein which functionally substitutes for said recombinant Rhabdovirus glycoprotein gene.

9. A recombinant Rhabdovirus that expresses a functional HIV envelope protein wherein said recombinant Rhabdovirus is replication-competent.

10. The recombinant Rhabdovirus of claim 9, wherein said Rhabdovirus is a recombinant rabies virus or a recombinant vesicular stomatitis virus.

11. The recombinant Rhabdovirus of claim 9, wherein said HIV envelope protein is from any HIV-1 isolate.

12. An immunogenic composition comprising a recombinant Rhabdovirus vector as in any one of claims 1 to 9 and an adjuvant.

13. A recombinant Ψ gene deficient rabies virus comprising a heterologous nucleic acid segment encoding an immunodeficiency virus envelope protein, or a subunit thereof.

14. The recombinant Ψ gene deficient rabies virus of claim 13, wherein said Rhadovirus is a rabies virus.

15. The recombinant Ψ gene deficient rabies virus of claim 13, wherein said immunodeficiency virus envelope protein, or a subunit thereof, is from a human immunodeficiency virus.

16. The recombinant Ψ gene deficient rabies virus of claim 13, wherein said immunodeficiency virus envelope protein, or a subunit thereof, is from a simian immunodeficiency virus.

17. A method of inducing an immunological response in a mammal, comprising:
a) delivering to a tissue of said mammal a recombinant Rhabdovirus vector that expresses a functional immunodeficiency virus envelope protein, or a subunit thereof, effective to induce an immunological response to said envelope protein;
b) expressing said envelope protein, or the subunit thereof, in vivo;
c) boosting said mammal by delivering an effective dose of an isolated immunodeficiency virus envelope protein, or a subunit thereof, in an adjuvant or by delivering an effective dose of a boost vaccine vector; and
d) inducing a neutralizing antibody response and/or long lasting cellular immune response thereto to protect said mammal from an immunodeficiency virus.

18. The method of claim 17, wherein said recombinant Rhabdovirus comprises a rabies virus genome.

19. The method of claim 18, wherein said rabies virus genome is deficient in Ψ gene.

20. The method of claim 18, wherein said rabies virus genome is deficient in a rabies virus glycoprotein gene.

21. The method of claim 19, wherein said rabies virus genome has glycoprotein gene from another class of Rhabdovirus in place of a rabies virus glycoprotein.

22. A method of inducing an immunological response in a mammal, comprising:
a) delivering to a tissue of said mammal a non-segmented negative-stranded RNA virus that expresses a functional immunodeficiency virus envelope protein, or a subunit thereof, effective to induce an immunological response to said envelope protein;
b) expressing said envelope protein, or the subunit thereof, in vivo;
c) boosting said mammal by delivering an effective dose of an isolated immunodeficiency virus envelope protein, or a subunit thereof, in an adjuvant or by delivering an effective dose of a boost vaccine vector; and
d) inducing a neutralizing antibody response and/or long lasting cellular immune response thereto to protect said mammal from an immunodeficiency virus.

23. The method of claim 22, wherein said non-segmented negative-stranded RNA virus is a Rabies virus or a Vesicular Stomatitis virus.

24. A recombinant non-segmented negative-stranded RNA virus vector comprising:
a) a modified negative-stranded RNA virus genome that is deficient in Ψ gene;
b) a new transcription unit that is inserted into said modified negative-stranded RNA virus genome to express heterologous nucleic acid sequences; and
c) a heterologous viral nucleic acid sequence that is inserted into said new transcription unit, wherein said recombinant non-segmented negative-stranded RNA virus vector is replication competent, and said heterologous viral nucleic acid sequence encodes an antigenic polypeptide.

25. A method of treating a mammal infected with an immunodeficiency virus, comprising:
a) administering to said mammal a non-segmented negative-stranded RNA virus that expresses a functional immunodeficiency virus envelope protein, or subunit thereof;
b) expressing said functional immunodeficiency virus envelope protein, or subunit thereof;
c) boosting said mammal by delivering an effective dose of an isolated immunodeficiency virus envelope protein, or a subunit thereof, in an adjuvant or by delivering an effective dose of a boost vaccine vector; and
d) inducing a neutralizing antibody response and/or long lasting cellular immune response to said functional immunodeficiency virus envelope protein, or subunit thereof.

26. The method of claim 25, wherein said immunodeficiency virus is any HIV-1 virus.

27. The method of claim 25, wherein said non-segmented negative-stranded RNA virus is a Rhabdovirus.

28. The method of claim 25, further comprising an induction of mucosal immunity to said functional immunodeficiency virus envelope protein, or subunit thereof.

29. The method of claim 25, wherein said long-lasting cellular response further comprises a cross-reactive CTL response wherein said cross-reactive CTLs are directed against envelope proteins, or subunits thereof, from different immunodeficiency virus strains.

30. A method of protecting a mammal from an immunodeficiency virus infection, comprising:
a) administering to said mammal a non-segmented negative-stranded RNA virus that expresses a functional immunodeficiency virus envelope protein, or subunit thereof;
b) expressing said functional immunodeficiency virus envelope protein, or subunit thereof;
c) boosting said mammal by delivering an effective dose of an isolated immunodeficiency virus envelope protein, or a subunit thereof, in an adjuvant or by delivering an effective dose of a boost vaccine vector; and
d) inducing a neutralizing antibody response and/or long lasting cellular immune response to said functional immunodeficiency virus envelope protein, or subunit thereof.

31. The method of claim 30, wherein said immunodeficiency virus is any HIV-l virus.

32. The method of claim 30, wherein said non-segmented negative-stranded RNA virus is a Rhabdovirus.

33. The method of claim 30, further comprising an induction of mucosal immunity to said functional immunodeficiency virus envelope protein, or subunit thereof.

34. The method of claim 30, wherein said long-lasting cellular response further comprises a cross-reactive CTL response wherein said cross-reactive CTLs are directed against envelope proteins, or subunits thereof, from different immunodeficiency virus strains.

35. A recombinant Rhabdovirus vector comprising:
a) a modified Rhabdovirus genome;
b) a new transcription unit inserted into the Rhabdovirus genome to express heterologous nucleic acid sequences; and
c) a heterologous viral nucleic acid sequence that is inserted into said new transcription unit, wherein said recombinant Rhabdovirus vector is replication competent, and said heterologous viral nucleic acid sequence encodes HCV E1, E2, and p7 antigenic polypeptides.

36. A recombinant Rhabdovirus vector comprising:
a) a modified Rhabdovirus genome;
b) a new transcription unit inserted into the Rhabdovirus genome to express heterologous nucleic acid sequences; and
c) a heterologous viral nucleic acid sequence that is inserted into said new transcription unit, wherein said recombinant Rhabdovirus vector is replication competent, and said heterologous viral nucleic acid sequence encodes an ectodomain of HCV E2 that has an amino acid deletion at its carboxy-terminus fused to a transmembrane domain and a cytoplasmic domain of human CD4, wherein a chimeric E2 antigenic polypeptide is expressed.

37. A recombinant Rhabdovirus vector comprising:
a) a modified Rhabdovirus genome;
b) a new transcription unit inserted into the Rhabdovirus genome to express heterologous nucleic acid sequences; and
c) a heterologous viral nucleic acid sequence that is inserted into said new transcription unit, wherein said recombinant Rhabdovirus vector is replication competent, and said heterologous viral nucleic acid sequence encodes an ectodomain of HCV E2 that has an amino acid deletion at its carboxy-terminus fused to a transmembrane domain of human CD4 and a cytoplasmic domain of a Rhabdovirus glycoprotein wherein a chimeric E2 antigenic polypeptide is expressed.

38. A recombinant Rhabdovirus that expresses a functional HCV envelope protein wherein said recombinant Rhabdovirus is replication-competent.

39. An immunogenic composition comprising a recombinant Rhabdovirus vector as in any one of claims 35 to 38 and an adjuvant.

40. A recombinant Ψ gene deficient rabies virus comprising a heterologous nucleic acid segment encoding a hepatitis C virus envelope protein, or a subunit thereof.

41. A method of inducing an immunological response in a mammal, comprising:
a) delivering to a tissue of said mammal a recombinant non-segmented negative stranded RNA virus vector that expresses a functional hepatitis C virus envelope protein, or a subunit thereof, in an amount effective to induce an immunological response to said envelope protein;
b) expressing said envelope protein, or the subunit thereof, in vivo;
c) boosting said mammal by delivering an effective dose of an isolated heptatits C virus envelope protein, or a subunit thereof, in an adjuvant or by delivering an effective dose of a boost vaccine vector; and
d) inducing a neutralizing antibody response and/or long lasting cellular immune response thereto to protect said mammal from a heptatits C virus.

42. The method of claim 41, wherein said recombinant Rhabdovirus is deficient in Ψ gene.

43. The method of claim 42, wherein said recombinant Rhabdovirus comprises a rabies virus genome.

44. A method of inducing an immunological response in a mammal, comprising:
a) delivering to a tissue of said mammal a recombinant non-segmented negative stranded RNA virus vector that expresses a functional hepatitis C virus envelope protein, or a subunit thereof, in an amount effective to induce an immunological response to said envelope protein;
b) expressing said envelope protein, or the subunit thereof, in vivo; and
c) inducing a long lasting cellular immune response thereto to protect said mammal from a heptatits C virus.

45. The method of claim 44, wherein said recombinant Rhabdovirus is deficient in Ψ gene.

46. The method of claim 46, wherein said recombinant Rhabdovirus comprises a rabies virus genome.

47. The method of claim 41 or 44 wherein said non-segmented negative stranded RNA virus comprises a Rhabdovirus vector.

48. A method of treating a mammal infected with a hepatitis C virus, comprising:
a) administering to said mammal a non-segmented negative-stranded RNA virus that expresses a functional hepatitic C virus envelope protein, or subunit thereof;
b) expressing said functional hepatitic C virus envelope protein, or subunit thereof;
c) boosting said mammal by delivering an effective dose of an isolated hepatitic C virus envelope protein, or a subunit thereof, in an adjuvant or by delivering an effective dose of a boost vaccine vector; and
d) inducing a neutralizing antibody response and/or long lasting cellular immune response to said functional hepatitic C virus envelope protein, or subunit thereof.

49. A method of treating a mammal infected with a hepatitis C virus, comprising:
a) administering to said mammal a non-segmented negative-stranded RNA virus that expresses a functional hepatitic C virus envelope protein, or subunit thereof;
b) expressing said functional hepatitic C virus envelope protein, or subunit thereof; and
c) inducing a long lasting cellular immune response to said functional hepatitic C virus envelope protein, or subunit thereof.

50. The method of claim 48 or 49, wherein said non-segmented negative-stranded RNA virus is a Rhabdovirus.

51. A method of protecting a mammal from a hepatitis C virus infection, comprising:
a) administering to said mammal a non-segmented negative-stranded RNA virus that expresses a functional hepatitis C virus envelope protein, or subunit thereof;
b) expressing said functional hepatitis C virus envelope protein, or subunit thereof;
c) boosting said mammal by delivering an effective dose of an isolated hepatitis C virus envelope protein, or a subunit thereof, in an adjuvant or by delivering an effective dose of a boost vaccine vector; and
d) inducing a neutralizing antibody response and/or long lasting cellular immune response to said functional hepatitis C virus envelope protein, or subunit thereof.

52. A method of protecting a mammal from a hepatitis C virus infection, comprising:
a) administering to said mammal a non-segmented negative-stranded RNA virus that expresses a functional hepatitis C virus envelope protein, or subunit thereof;
b) expressing said functional hepatitis C virus envelope protein, or subunit thereof; and
c) inducing a long lasting cellular immune response to said functional hepatitis C virus envelope protein, or subunit thereof.

53. A method of inducing an immunological response in a mammal, comprising:
a) delivering to a tissue of said mammal a recombinant non-segmented negative stranded RNA virus virion wherein said virion has on its surface a functional hepatitis C virus envelope protein, or a subunit thereof, effective to induce an immunological response to said envelope protein;
b) expressing said envelope protein, or the subunit thereof, in vivo;
c) boosting said mammal by delivering an effective dose of an isolated heptatits C virus envelope protein, or a subunit thereof, in an adjuvant or by delivering an effective dose of a boost vaccine vector; and
d) inducing a neutralizing antibody response and/or long lasting cellular immune response thereto to protect said mammal from a heptatits C virus.

54. The method of claim 53, wherein said recombinant Rhabdovirus is deficient in Ψ gene.

55. The method of claim 54, wherein said recombinant Rhabdovirus comprises a rabies virus genome.

56. A method of inducing an immunological response in a mammal, comprising:
a) delivering to a tissue of said mammal a recombinant non-segmented negative stranded RNA virus virion wherein said virion has on its surface a functional hepatitis C virus envelope protein, or a subunit thereof, effective to induce an immunological response to said envelope protein;
b) expressing said envelope protein, or the subunit thereof, in vivo; and
c) inducing a long lasting cellular immune response thereto to protect said mammal from a heptatits C virus.

57. The method of claim 56, wherein said recombinant Rhabdovirus is deficient in Ψ gene.

58. The method of claim 57, wherein said recombinant Rhabdovirus comprises a rabies virus genome.

59. The method of claim 53 or 56 wherein said non-segmented negative stranded RNA virus comprises a Rhabdovirus vector.

60. A method of treating a mammal infected with a hepatitis C virus, comprising:
a) administering to said mammal a non-segmented negative-stranded RNA virus virion wherein said virion has on its surface a functional hepatitic C virus envelope protein, or subunit thereof;
b) boosting said mammal by delivering an effective dose of an isolated hepatitic C virus envelope protein, or a subunit thereof, in an adjuvant or by delivering an effective dose of a boost vaccine vector; and
c) inducing a neutralizing antibody response and/or long lasting cellular immune response to said functional hepatitic C virus envelope protein, or subunit thereof.

61. A method of treating a mammal infected with a hepatitis C virus, comprising:
a) administering to said mammal a non-segmented negative-stranded RNA virus virion wherein said virion has on its surface a functional hepatitic C virus envelope protein, or subunit thereof; and
b) inducing a long lasting cellular immune response to said functional hepatitic C virus envelope protein, or subunit thereof.

62. The method of claim 60 or 61, wherein said non-segmented negative-stranded RNA virus is a Rhabdovirus.

63. A method of protecting a mammal from a hepatitis C virus infection, comprising:
a) administering to said mammal a non-segmented negative-stranded RNA virus virion wherein said virion has on its surface a functional hepatitis C virus envelope protein, or subunit thereof;
b) boosting said mammal by delivering an effective dose of an isolated hepatitis C virus envelope protein, or a subunit thereof, in an adjuvant or by delivering an effective dose of a boost vaccine vector; and
c) inducing a neutralizing antibody response and/or long lasting cellular immune response to said functional hepatitis C virus envelope protein, or subunit thereof.

64. A method of protecting a mammal from a hepatitis C virus infection, comprising:
a) administering to said mammal a non-segmented negative-stranded RNA virus virion wherein said virion has on its surface a functional hepatitis C virus envelope protein, or subunit thereof; and
b) inducing a long lasting cellular immune response to said functional hepatitis C virus envelope protein, or subunit thereof.

65. A method for diagnosing a patient with a hepatitis C infection, comprising:
a) contacting an immobilized immunoassay reagent comprising an antigenic peptide of hepatitis C virus with a biological sample from said patient suspected of a hepatitis C infection under conditions such that any immunospecific binding occurs; and
b) detecting or measuring an amount of said immunospecific binding by antibodies in said biological sample from said patient that are bound to said reagent, wherein detection of said antibodies indicates said hepatitis C infection.

66. The method of claim 65 wherein said antigenic peptide of hepatitis C virus is an E2 antigen, or subunit thereof.

67. A method for diagnosing a patient with a hepatitis C infection, comprising:
a) contacting an immobilized immunoassay reagent comprising an anti-hepatitis C antibody(s) with a biological sample from said patient suspected of a hepatitis C infection under conditions such that any immunospecific binding occurs; and
b) detecting or measuring an amount of said immunospecific binding by hepatitis C virus and/or viral antigens in said biological sample from said patient that are bound to said reagent, wherein detection of said hepatitis C virus and/or viral antigens indicates said hepatitis C infection.

68. The method of claim 67 wherein said immobilized immunoassay reagent comprises a polyclonal or monoclonal antibody that is capable of binding to and detecting said HCV virus and/or viral antigens in said biological sample of said patient.

69. A recombinant Rhabdovirus vector comprising:
a) a modified Rhabdovirus genome;
b) a new transcription unit inserted into the Rhabdovirus genome to express heterologous nucleic acid sequences; and
c) a heterologous viral nucleic acid sequence that is inserted into said new transcription unit, wherein the recombinant Rhabdovirus vector is replication competent, and said heterologous viral nucleic acid sequence encodes an HIV gag antigenic polypeptide.

70. A recombinant Rhabdovirus that expresses a functional HIV gag protein wherein said recombinant Rhabdovirus is replication-competent.

71. An immunogenic composition comprising a recombinant Rhabdovirus vector of claim 69 or 70.

72. A method of inducing an immunological response in a mammal, comprising:
a) delivering to a tissue of said mammal a recombinant non-segmented negative stranded RNA virus vector that expresses a functional immunodeficiency virus gag protein, or a subunit thereof, in an amount effective to induce an immunological response to said gag protein;
b) expressing said gag protein, or the subunit thereof, in vivo;
c) boosting said mammal by delivering an effective dose of an isolated gag protein, or a subunit thereof, in an adjuvant or by delivering an effective dose of a boost vaccine vector; and
d) inducing a neutralizing antibody response and/or long lasting cellular immune response thereto to protect said mammal from an immunodeficiency virus.

73. The method of claim 72, wherein said recombinant Rhabdovirus comprises a rabies virus genome.

74. A method of inducing an immunological response in a mammal, comprising:
a) delivering to a tissue of said mammal a non-segmented negative-stranded RNA virus that expresses a functional immunodeficiency virus gag protein, or a subunit thereof, effective to induce an immunological response to said gag protein;
b) expressing said gag protein, or the subunit thereof, in vivo;
c) boosting said mammal by delivering an effective dose of an isolated immunodeficiency virus gag protein, or a subunit thereof, in an adjuvant or by delivering an effective dose of a boost vaccine vector; and
d) inducing a neutralizing antibody response and/or long lasting cellular immune response thereto to protect said mammal from an immunodeficiency virus.

75. The method of claim 74, wherein said non-segmented negative-stranded RNA virus is a Rabies virus.

76. A method of treating a mammal infected with an immunodeficiency virus, comprising:
a) administering to said mammal a non-segmented negative-stranded RNA virus that expresses a functional immunodeficiency virus gag protein, or subunit thereof;
b) expressing said functional immunodeficiency virus gag protein, or subunit thereof;
c) boosting said mammal by delivering an effective dose of an isolated immunodeficiency virus gag protein, or a subunit thereof, in an adjuvant or by delivering an effective dose of a boost vaccine vector; and
d) inducing a neutralizing antibody response and/or long lasting cellular immune response to said functional immunodeficiency virus gag protein, or subunit thereof.

77. The method of claim 76, wherein said non-segmented negative-stranded RNA virus is a Rhabdovirus.

78. The method of claim 76, further comprising an induction of mucosal immunity to said functional immunodeficiency virus gag protein, or subunit thereof.

79. The method of claim 76, wherein said long-lasting cellular response further comprises a cross-reactive CTL response wherein said cross-reactive CTLs are directed against gag proteins, or subunits thereof, from different immunodeficiency virus strains.

80. A method of protecting a mammal from an immunodeficiency virus infection, comprising:
a) administering to said mammal a non-segmented negative-stranded RNA virus that expresses a functional immunodeficiency virus gag protein, or subunit thereof;
b) expressing said functional immunodeficiency virus gag protein, or subunit thereof;
c) boosting said mammal by delivering an effective dose of an isolated immunodeficiency virus gag protein, or a subunit thereof, in an adjuvant or by delivering an effective dose of a boost vaccine vector; and
d) inducing a neutralizing antibody response and/or long lasting cellular immune response to said functional immunodeficiency virus gag protein, or subunit thereof.

81. The method of claim 80, wherein said non-segmented negative-stranded RNA virus is a Rhabdovirus.

82. The method of claim 80, further comprising an induction of mucosal immunity to said functional immunodeficiency virus gag protein, or subunit thereof.

83. The method of claim 80, wherein said long-lasting cellular response further comprises a cross-reactive CTL response wherein said cross-reactive CTLs are directed against gag proteins, or subunits thereof, from different immunodeficiency virus strains.


SEQUENCE LISTING (21 sequences)

SEQ ID NO: 1 - DNA, 33 bp, Artificial Sequence
  primer
cctcaaaagaccccgggaaagatggttcctcag

SEQ ID NO: 2 - DNA, 36 bp, Artificial Sequence
  primer
gactgtaaggacyggctagcctttcaacgatccaag

SEQ ID NO: 3 - DNA, 35 bp, Artificial Sequence
  primer
ttttgctagcttataaagtgctgggtcatctaagc

SEQ ID NO: 4 - DNA, 33 bp, Artificial Sequence
  primer
cactacaagtcagtcgagacttggaatgagatc

SEQ ID NO: 5 - DNA, 24 bp, Artificial Sequence
  primer
tctcgagtgttctctctccaacaa

SEQ ID NO: 6 - DNA, 64 bp, Artificial Sequence
  primer
aagctagcaaaacgtacgggaggggtgttagtttttttcatggacttggatcgttgaaaggacg

SEQ ID NO: 7 - DNA, 45 bp, Artificial Sequence
  primer
gggctgcagctcgagcgtacgaaaatgagagtgaaggagatcagg

SEQ ID NO: 8 - DNA, 30 bp, Artificial Sequence
  primer
cctctagattatagcaaagccctttccaag

SEQ ID NO: 9 - DNA, 42 bp, Artificial Sequence
  synthetic oligonucleotide primer
gtgagaccagactgtaattaattaacgtcctttcaacgatcc

SEQ ID NO: 10 - DNA, 34 bp, Artificial Sequence
  synthetic oligonucleotide primer
ctcgagcgtacgaaaatgaattccgacctcatgg

SEQ ID NO: 11 - DNA, 30 bp, Artificial Sequence
  synthetic oligonucleotide primer
cgttaagctagctcatgcgtatgcccgctg

SEQ ID NO: 12 - DNA, 33 bp, Artificial Sequence
  synthetic oligonucleotide primer
ctcgagcgtacgaaaatggtcctggtagtgctg

SEQ ID NO: 13 - DNA, 32 bp, Artificial Sequence
  synthetic oligonucleotide primer
aattgctagctcaaatggggctacatgtcttc

SEQ ID NO: 14 - DNA, 33 bp, Artificial Sequence
  synthetic oligonucleotide primer
cccgggttaacagaagagtcaatcgatcagaac

SEQ ID NO: 15 - DNA, 29 bp, Artificial Sequence
  synthetic oligonucleotide primer
cctctagattacagtctggtctcaccccc

SEQ ID NO: 16 - DNA, 21 bp, Artificial Sequence
  synthetic oligonucleotide primer
aacgaagaagatgcctagccc

SEQ ID NO: 17 - protein, 20 aa, Artificial Sequence
  synthetic peptide
EATYSRCGSGPWITPRCMVD

SEQ ID NO: 18 - protein, 9 aa, Artificial Sequence
  synthetic peptide
AMQMLKETI

SEQ ID NO: 19 - DNA, 42 bp, Artificial Sequence; identical to SEQ ID NO: 9
  synthetic oligonucleotide primer

SEQ ID NO: 20 - DNA, 37 bp, Artificial Sequence
  synthetic oligonucleotide primer
aaactcgagcgtacgacaatgggtgcgagagcgtcag

SEQ ID NO: 21 - DNA, 42 bp, Artificial Sequence
  synthetic oligonucleotide primer
aaagctagcttaattaatcgcgattattgtgacgaggggtcg